(19) United States
(12) Patent Application Publication
(10) Pub. No.: US 2024/0315971 A1
(43) Pub. Date: Sep. 26, 2024

(54) MICROSPHERE FORMULATIONS AND METHODS OF PREPARATION THEREOF

(71) Applicant: Lotus BioPharma Inc., Monmouth Junction, NJ (US)

(72) Inventors: Chao Li, Monmouth Junction, NJ (US); Nancy L. Li, Bayside, NY (US); Kingteh Lin, Monmouth Junction, NJ (US)

(73) Assignee: Lotus BioPharma Inc., Monmouth Junction, NJ (US)

(21) Appl. No.: 18/592,719

(22) Filed: Mar. 1, 2024

Related U.S. Application Data
(60) Provisional application No. 63/488,300, filed on Mar. 3, 2023.

(51) Int. Cl. A61K 9/16; A61K 31/137; A61K 31/18; A61K 31/216; A61K 31/4025; A61K 31/426; A61K 31/4725

(52) U.S. Cl. CPC A61K 9/1647; A61K 9/1694; A61K 31/137; A61K 31/18; A61K 31/216; A61K 31/4025; A61K 31/426; A61K 31/4725

(57) ABSTRACT

This disclosure provides injectable microsphere formulations capable of controlled long-term sustained release. The disclosed microsphere formulations demonstrate a zero-order release of active agents over a predetermined period of time. Also disclosed are methods of preparing the microsphere formulations and methods of use thereof. The disclosed methods can be readily scaled up while maintaining a high drug encapsulation efficiency.

### CROSS-REFERENCE TO RELATED APPLICATIONS

This application claims priority under 35 U.S.C. § 119(e) to U.S. Provisional Patent Application No. 63/488,300, filed Mar. 3, 2023. The foregoing application is incorporated by reference herein in its entirety.

### FIELD OF THE INVENTION

This invention relates generally to microsphere-based formulations, methods of preparation thereof, and methods of use thereof.

### BACKGROUND OF THE INVENTION

Overactive bladder (OAB) is a chronic medical condition with characteristic symptoms of urinary urgency, frequency, and nocturia, with or without urgency incontinence, in the absence of urinary tract infection or other obvious pathology. It has a tremendous impact on the quality of life of both men and women, such as the performance of daily life activities, social functions, physical exercise, sleep, and sexual function. Overactive bladder affects nearly all groups of the population and more than 33 million adult Americans.

The treatment for overactive bladder includes education, lifestyle changes, bladder training, pelvic floor muscle training, and anticholinergic or antimuscarinic medications. Additional treatments include pessary placement and surgery. Although some patients can benefit from conservative measures such as lifestyle changes and retraining, pharmaceutical treatments are ultimately required. One such treatment is oxybutynin, an anticholinergic and antimuscarinic agent. Its anticholinergic activities contribute to oxybutynin's medical usefulness. Still, it is also the culprit of certain uncomfortable side effects, such as dry mouth, difficulty in micturition, constipation, blurred vision, drowsiness, dizziness, etc. These side effects are dose-related and mainly caused by oxybutynin's primary active metabolite, N-desethyloxybutynin. In fact, the above-mentioned side effects are observed in most patients using oral therapy. In many cases, these side effects are severe enough to cause the patients to discontinue their treatment. Although the oxybutynin transdermal preparations with lower blood levels and a steady blood concentration have improved anticholinergic adverse event profile due to bypassing the first pass metabolism system, they still have anticholinergic adverse events in addition to the commonly seen application site reactions.

Therefore, there is a need to develop an improved formulation that can bypass first-pass metabolism, while having controlled long-acting sustained release with low toxicity and fewer adverse events.

### SUMMARY OF THE INVENTION

This disclosure addresses the need mentioned above in a number of aspects by providing a novel microsphere-based formulation (e.g., injectable microspheres) that can bypass first-pass metabolism and has controlled long-acting sustained release with low toxicity, little to no adverse events, and pharmaceutical effects not inferior to the currently used non-microsphere-based formulations. The disclosed microsphere formulation can achieve better treatment outcomes and patient medical compliance.

In one aspect, this disclosure provides a microsphere for controlled long-term sustained delivery of an active agent. In some embodiments, the microsphere comprises the active agent and a polymer carrier that encapsulates the active agent, wherein the polymer carrier comprises poly(lactide-co-glycolide) (PLGA), polylactide (PLA), polyglycolic acid (PLG), polyethylene glycol-PLA, PLA-polycaprolactone (PCL), a polyorthoester, a polyphosphazene, a polyphosphoester, or a combination thereof. In some embodiments, the polymer carrier comprises PLGA, PLA, or a combination thereof.

In some embodiments, the microsphere has a diameter of from about 8 μm to about 75 μm.

In some embodiments, the microsphere comprises from about 4% (w/w) to about 50% (w/w) of the active agent (e.g., oxybutynin, tolterodine, solifenacin, darifenacin, pramipexole, or tamsulosin) by weight of the microsphere.

In some embodiments, the polymer carrier is biodegradable and biocompatible. In some embodiments, the polymer carrier has a degradation half-life of at least 2 months under physiological conditions. In some embodiments, the microsphere has a zero-order release when contacting with an aqueous phase. In some embodiments, the microsphere maintains a controlled and sustained release for a period from 1 week to 6 months.

In some embodiments, the active agent comprises oxybutynin, tolterodine, solifenacin, darifenacin, pramipexole, and/or tamsulosin. In some embodiments, the active agent comprises a free base, a stereoisomer, a derivative, an analog, a prodrug, or a pharmaceutically acceptable salt of oxybutynin, tolterodine, solifenacin, darifenacin, pramipexole, or tamsulosin. In some embodiments, the active agent comprises oxybutynin; or a free base, a stereoisomer, a derivative, an analog, a prodrug, or a pharmaceutically acceptable salt of oxybutynin.

In another aspect, this disclosure provides a composition comprising the microsphere described herein. In some embodiments, the composition further comprises at least one anti-cryogenic agent. In some embodiments, the composition further comprises an excipient.

In some embodiments, the composition further comprises an additional therapeutic agent. In some embodiments, the additional therapeutic agent comprises an α2δ subunit calcium channel modulator, a β3 adrenergic agonist, a spasmolytic, a neurokinin receptor antagonist, a bradykinin receptor antagonist, a nitric oxide donor, or a combination thereof.

Also within the scope of this disclosure is a kit or an implant. In some embodiments, the kit or implant comprises the microsphere or the composition thereof, as described herein.

In yet another aspect, this disclosure provides a method of preparing a microsphere described herein. In some embodiments, the method comprises:
(a) dissolving a polymer carrier in at least one organic solvent that is substantially non-miscible with water to form a polymer solution;
(b) dissolving an active agent in the polymer solution by single emulsion/solvent extraction to obtain an oil phase drug-polymer solution, or by double emulsion to obtain a water-in-oil (W/O) primary emulsion, and cooling the oil phase drug-polymer solution or the water-in-oil primary emulsion to about 45° F.;
(c) adding the oil phase drug-polymer solution or the water-in-oil primary emulsion to a PVA solution, wherein the PVA solution is in the form of an aqueous, continuous phase;
(d) homogenizing the oil phase drug-polymer solution or the water-in-oil primary emulsion with the 1% PVA solution at a predetermined mixing rate to obtain a microsphere suspension having semi-solid microencapsulated droplets, wherein the microsphere suspension is in the form of oil-in-water (O/W) or water-in-oil-in-water (W/O/W) emulsion;
(e) extracting the organic solvent by:
placing a container containing water precooled to about 45° F. in an ice-cooled water bath,
transferring the microsphere suspension into the container while stirring the microsphere suspension with a mixer,
maintaining the container in the ice-cooled water bath for about 3 hours to allow the microsphere suspension to harden and the temperature thereof to rise from about 45° F. to about 55° F., and
removing the ice-cooled water bath and exposing the microsphere suspension to an ambient temperature without heating equipment for about 1 hour, such that the temperature of the hardened microsphere suspension reaches about 66° F.; and
(f) collecting microspheres by screening through a mesh sieve, washing with water through a filter, and re-suspending in a D-mannitol solution for freeze-drying to obtain powdery lyophilized microspheres.

In some embodiments, the method further comprises: after step (f), quenching the powdery lyophilized microspheres one or more times at a temperature below 25 degrees Celsius. In some embodiments, the step of quenching is performed by placing the powdery lyophilized microspheres at −20 degrees Celsius.

In some embodiments, the organic solvent comprises dichloromethane.

In some embodiments, the polymer solution comprises from about 50 mg/mL to about 1200 mg/mL of the polymer carrier.

In some embodiments, the polymer carrier comprises PLGA, PLA, PLG, polyethylene glycol-PLA, PCL, a polyorthoester, a polyphosphazene, a polyphosphoester, or a combination thereof. In some embodiments, the polymer carrier comprises PLGA.

In some embodiments, the PVA solution comprises from about 0.1% (w/v) to about 5% (w/v) of PVA. In some embodiments, the PVA solution contains about 1% (w/v) of PVA.

In some embodiments, the PVA solution comprises a buffer with a concentration of about 0.0001 M to about 1 M. In some embodiments, the buffer has a concentration of about 0.05 M.

In some embodiments, the buffer has a pH of about 1 to about 14. In some embodiments, the buffer has a pH of about 8.1. In some embodiments, the buffer is a phosphate-buffered saline (PBS) buffer.

In some embodiments, the PVA solution at step (c) has a temperature of from about 35° F. to 73° F. In some embodiments, the PVA solution at step (c) has a temperature of about 45° F.

In some embodiments, the predetermined mixing rate is from about 2000 rpm to about 20000 rpm.

In some embodiments, an aqueous phase in step (e) has a volume at least 2 times greater than that of an oil/water phase.

In some embodiments, the aqueous phase in step (e) comprises a buffer with a concentration of about 0.001 M to about 2 M. In some embodiments, the buffer has a pH of from about 1 to about 14.

In some embodiments, during step (e), the temperature of the microsphere suspension rises from about 35° F. to the ambient temperature. In some embodiments, the ambient temperature is about 73° F.

In some embodiments, when the container is in the ice-cooled water bath, the temperature of the microsphere suspension gradually increases as that of the ice-cooled water bath does, and when the iced-cooled water bath is removed, the temperature of the microsphere suspension continues to gradually increase towards the ambient temperature.

In some embodiments, the microspheres have an average particle size of from about 1 μm to about 300 μm.

In some embodiments, the active agent comprises oxybutynin, tolterodine, solifenacin, darifenacin, pramipexole, and/or tamsulosin. In some embodiments, the active agent comprises a free base, a stereoisomer, a derivative, an analog, a prodrug, or a pharmaceutically acceptable salt of oxybutynin, tolterodine, solifenacin, darifenacin, pramipexole, or tamsulosin. In some embodiments, the active agent comprises oxybutynin; or a free base, a stereoisomer, a derivative, an analog, a prodrug, or a pharmaceutically acceptable salt of oxybutynin.

In another aspect, this disclosure further provides a method of treating or preventing a lower urinary tract disorder or reducing muscle spasms of bladder and urinary tract in a subject in need thereof. In some embodiments, the method comprises administering to the subject an effective amount of the microsphere or the composition, as described herein.

In yet another aspect, this disclosure also provides a method of reducing muscle spasms of bladder and urinary tract in a subject in need thereof. In some embodiments, the method comprises administering to the subject an effective amount of the microsphere or the composition, as described herein.

In some embodiments, the lower urinary tract disorder is characterized by at least one symptom selected from the group consisting of urinary frequency, urinary urgency, and nocturia.

In some embodiments, the microsphere or the composition is administered to the subject intramuscularly or subcutaneously.

In some embodiments, the microsphere or the composition is administered to the subject as part of an implant.

In some embodiments, the microsphere or the composition maintains a sustained release of the active agent (e.g., oxybutynin, tolterodine, solifenacin, darifenacin, pramipexole, or tamsulosin) from about 4 weeks to about 18 weeks (e.g., 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks) in the subject.

In some embodiments, the effective amount of the microsphere or the composition is from about 0.5 mg/kg to about 8 mg/kg (e.g., 0.5 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg) by body weight of the subject.

In some embodiments, the method further comprises administering to the subject a second therapeutic agent.

In some embodiments, the second therapeutic agent comprises an α2δ subunit calcium channel modulator, a β3 adrenergic agonist, a spasmolytic, a neurokinin receptor antagonist, a bradykinin receptor antagonist, a nitric oxide donor, or a combination thereof. In some embodiments, the α2δ subunit calcium channel modulator comprises gabapentin, pregabalin, or a combination thereof.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combinations of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows the in vitro release profiles of oxybutynin microspheres prepared according to Example 7.

FIG. 2 shows the in vitro release profiles of oxybutynin microspheres from a 10× scaled-up preparation compared to the original preparation without scaling up.

FIG. 3 shows in vitro release profiles of a 16-week formulation prepared as described in Example 23.

FIG. 4 shows the in vitro release profiles of pramipexole microspheres as described in Example 29.

### DETAILED DESCRIPTION OF THE INVENTION

This disclosure provides a novel microsphere-based formulation (e.g., an injectable microsphere) for active agents that can bypass first-pass metabolism and has controlled long-acting sustained release with low toxicity, little to no adverse events, and pharmaceutical effects not inferior to the currently used non-microsphere-based formulations. The disclosed microsphere formulations demonstrate a zero-order release of active agents, with R² of 0.997, and 98-100% of active agents being released from the microspheres over 5-7 weeks and with R² of 0.996, and 98-100% of active agents being released from the microspheres over 15-17 weeks. Also disclosed are methods of preparing the microsphere formulations and methods of use thereof. The disclosed methods can be readily scaled up while maintaining at least a 95±2% high drug encapsulation efficiency.

### Microsphere-Based Formulations

In one aspect, this disclosure provides a microsphere for controlled long-term sustained delivery of an active agent. In some embodiments, the microsphere may include the active agent and a polymer carrier that encapsulates the active agent.

As used herein, the term “active agent” refers to any molecule, compound, or composition. For the methods and compositions described herein, any active agent can be maintained within a microsphere formulation. Examples of active agents include, but are not limited to, small molecules, proteins, peptides, antigens, immunogens, vaccines, antibodies or portions thereof (e.g., antibody-like molecules), enzymes, nucleic acids (e.g., oligonucleotides, polynucleotides, siRNA, shRNA), aptamers, viruses, bacteria, cells, photo synthetic and energy-harvesting compounds, flavors, antibiotics, therapeutic agents, diagnostic agents such as contrast agents or dye, viral vectors, and anti-venom.

“Small molecule” refers to any organic material that is not a polymer, and “small molecules” may actually be quite large. Small molecules may include repeat units in some circumstances.

In some embodiments, the active agent may be selected from active agent selected from the group consisting of peptides, nucleotides, anti-obesity drugs, nutraceuticals, corticosteroids, elastase inhibitors, analgesics, anti-fungals, oncology therapies, anti-emetics, analgesics, cardiovascular agents, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives, astringents, beta-adrenoceptor blocking agents, blood products and substitutes, cardiac inotropic agents, contrast media, corticosteroids, cough suppressants, diagnostic agents, diagnostic imaging agents, diuretics, dopaminergics, haemostatics, immuriological agents, lipid regulating agents, muscle relaxants, parasympathomimetics, parathyroid calcitonin and biphosphonates, prostaglandins, radio-pharmaceuticals, sex hormones, anti-allergic agents, stimulants and anoretics, sympathomimetics, thyroid agents, vasodilators and xanthines.

In some embodiments, the active agent comprises oxybutynin, a stereoisomer thereof, a derivative thereof, an analog thereof, a prodrug thereof, or a pharmaceutically acceptable salt thereof.

### Microspheres

As used herein, “microspheres” refer to polymers or combinations of polymers made into bodies of various sizes. The microspheres can be in any shape, although they can be in substantially spherical shape. In some embodiments, as part of the injectable composition of this disclosure, microspheres may also be sterilized before injection.

In some embodiments, microspheres are biodegradable. As used herein, “biodegradable” microspheres refer to microspheres that are capable of being absorbed by the body chemically, physiologically, or by other biological means over a period of time.

In some embodiments, the microsphere is provided as an injectable formulation. As used herein, “injectable” means that microspheres or compositions thereof can be administered, delivered, or carried into the body via syringes, catheters, needles, or other means for injecting or infusing microspheres in a liquid medium. For example, microspheres or compositions thereof may be injectable through needles of about 18 to 26 gauge, e.g., about 22 to 24 gauge, and microspheres are not capable of being digested or eliminated by macrophage or other elements of the mammal's immune or lymphatic system of a subject.

In some embodiments, microspheres may include one or more chemical modifications. As used herein, “chemical modification” refers to the changes in chemical properties and characteristics of the microspheres, either during their production process or by way of mixing or contacting them with various agents or tissues, such that the microspheres can perform, in addition to tissue bulking, other functions once injected into the body of a subject. For example, the microspheres may contain other chemicals within their structure or on their surfaces, thereby displaying particular properties, such as therapeutic, radio-pacifying, and contrasting effects, promotion of cell adhesion, and capability of being further chemically modified.

In some embodiments, microspheres may have any shape. In some embodiments, the microsphere may be substantially spherical. As used herein, “substantially spherical” refers to a shape close to a perfect sphere, defined as a volume that presents the lowest external surface area. Specifically, “substantially spherical” means that when viewing any particle cross-section, the difference between the average major diameter and the average minor diameter is less than 20%. The surfaces of the microspheres may appear smooth under magnification of up to 1000 times. The microspheres may include, in addition to the particles, other materials as described and defined herein.

In some embodiments, at least about 10%, 20%, 30%, 40%, 50%60%, 70%, 80% or 90% of microspheres can be about 0.5 μm to about 500 μm in the largest dimension (e.g., diameter), e.g., about 1 μm to about 300 μm, about 1 μm to about 200 μm, about 3 μm to about 150 μm, about 5 μm to about 100 μm, or about 8 μm to about 75 μm in its largest dimension.

In some embodiments, the microspheres may have the largest dimension (e.g., diameter) of less than about 100 μm. In some embodiments, the microspheres may have the largest dimension (e.g., diameter) of less than about 90 μm. In some embodiments, the microspheres may have the largest dimension of less than about 80 μm. In some embodiments, the microspheres may have the largest dimension of less than about 75 μm. In some embodiments, the microspheres may have the largest dimension of less than about 70 μm. In some embodiments, the microspheres may have the largest dimension of less than about 60 μm. In some embodiments, the microspheres may have the largest dimension of less than about 50 μm. In some embodiments, the microspheres may have the largest dimension of less than about 40 μm. In some embodiments, the microspheres may have the largest dimension of less than about 30 μm. In some embodiments, the microspheres may have the largest dimension of less than about 20 μm.

In some embodiments, the microspheres can have a diameter (e.g., average diameter or mean diameter) of between about 0.5 μm and about 500 μm. In some embodiments, the microspheres can have a diameter of between about 1 μm and about 300 μm. In some embodiments, the microspheres can have a diameter of between about 1 μm and about 200 μm. In some embodiments, the microspheres can have a diameter of between about 3 μm and about 150 μm. In some embodiments, the microspheres can have a diameter of between about 5 μm and about 100 μm. In some embodiments, the microspheres can have a diameter of between about 8 μm and about 75 μm.

In some embodiments, the microsphere has a diameter (e.g., average diameter or mean diameter) of from about 8 μm to about 75 μm (e.g., about 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 75 μm).

In some embodiments, the microsphere has a zero-order release when contacting with an aqueous phase. As used herein, the term “zero-order release,” “zero-order dissolution,” or “zero-order rate” refers to a rate of release of a drug from the solid dosage form after coming in contact with an aqueous environment, which is uniform or nearly uniform independent of the drug concentration in the dosage form during a given period. The disclosed microspheres with zero-order release generally enable reduced dosing frequency compared to less sustained or more unevenly released dosage forms, thus improving patient compliance. In addition, the disclosed microspheres with zero-order release generally provide maximum therapeutic value with minimal side effects.

In some embodiments, the microsphere maintains a controlled and sustained release for a period from 1 week to 6 months (e.g., about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, or 28 weeks).

As used herein, the term “controlled release” refers generally to delivery mechanisms that make an active ingredient available to the biological system of a host in a manner that supplies the drug according to a desired temporal pattern. As used herein, the term “sustained release” means that a pharmaceutical active agent is released over an extended period.

### Oxybutynin

Oxybutynin is also known by several IUPAC names such as α-cyclohexyl-α-hydroxy-benzenacetic acid 4-(diethylamino)-2-butynyl ester; α-phenylcyclo-hexyaneglycolic acid 4-(diethylamino)-2-butynyl ester; and 4-diethylamino-2-butynyl phenylcyclo-hexylglycolate. The oxybutynin acid addition salt, oxybutynin HCl, is listed in Merck Index, entry no., 7089, at page 1193, 12th ED., (1996). “Oxybutynin,” as used herein, includes oxybutynin free base and its salts, such as oxybutynin HCl, their analogs and related compounds, isomers, polymorphs, and prodrugs thereof. It is well known that oxybutynin may exist in one or both of its isomeric forms, known as the (R)- and (S)-isomers, or a mixture of these two isomers. Oxybutynin HCl is approved for use in treating overactive bladder with symptoms of urinary incontinence, urgency, and frequency in adults and neurogenic bladder in pediatric patients at or above six years of age.

The chemical structure of oxybutynin is shown below:

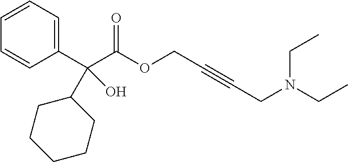 [SMILES: CCN(CC)CC#CCOC(=O)C(O)(c1ccccc1)C1CCCCC1]

In some embodiments, oxybutynin may include an oxybutynin free base, a stereoisomer thereof, a derivative thereof, an analog thereof, a prodrug thereof, or a pharmaceutically acceptable salt thereof.

Examples of active agents suitable for the disclosed microsphere formulations may include compounds similar to oxybutynin with comparable physicochemical properties, such as tolterodine, solifenacin, and darifenacin. These compounds are primarily used in the treatment of overactive bladder and exhibit muscarinic receptor antagonism, like oxybutynin.

Further examples of active agents suitable for the disclosed microsphere formulations may include the compounds with the below structure will have similar physicochemical properties to Oxybutynin. R1, R2, R3 can be any organic groups, including but not limited to: Methyl, ethyl, propenyl, iso-propyl. Butyl . . . , alcohols, heterocycle, amine, ester, etc. Any modifications to the original structure will fall into this category. The —OH group modification can affect its physical and chemical properties, such as solubility either in aqueous or organic solvent, density, melting point, etc., but not affect its function for the treatment.

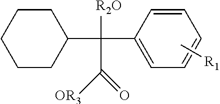

additional examples of active agents suitable for the disclosed microsphere formulations may include Pramipexole and Tamsulosin.

### Pramipexole

Pramipexole is used to treat Parkinson disease. It may be used alone or in combination with other medicines (e.g., levodopa). Pramipexole is a dopamine agonist that works on the nervous system to help treat the symptoms of Parkinson disease. Pramipexole is also used to treat Restless Legs Syndrome (RLS).

The chemical structure of Pramipexole is shown below:

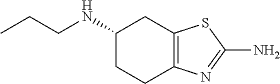

In some embodiments, Pramipexole may include a Pramipexole free base, a stereoisomer thereof, a derivative thereof, an analog thereof, a prodrug thereof, or a pharmaceutically acceptable salt thereof.

### Tamsulosin

Tamsulosin is used to treat men who have symptoms of an enlarged prostate gland, which is also known as benign enlargement of the prostate (benign prostatic hyperplasia or BPH). Benign enlargement of the prostate is a problem that can occur in men as they get older.

The chemical structure of Tamsulosin is shown below:

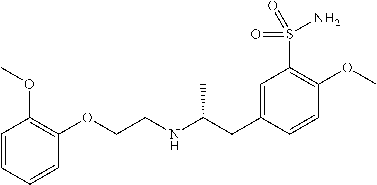 [SMILES: COc1ccccc1OCCN[C@H](C)Cc1ccc(OC)c(S(N)(=O)=O)c1]

In some embodiments, Tamsulosin may include a Tamsulosin free base, a stereoisomer thereof, a derivative thereof, an analog thereof, a prodrug thereof, or a pharmaceutically acceptable salt thereof.

In some embodiments, other alpha 1 blockers, such as alfuzosin, doxazosin, terazosin, tamsulosin, and prazosin can also be used in the disclosed microsphere formulations.

In some embodiments, the microsphere may include from about 1% (w/w) to about 75% (w/w) of the active agent (e.g., oxybutynin, tolterodine, solifenacin, darifenacin, pramipexole, or tamsulosin) by weight of the microsphere, e.g., about 4% (w/w) to about 50% (w/w) (e.g., 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75% (w/w)) of the active agent (e.g., oxybutynin, tolterodine, solifenacin, darifenacin, pramipexole, or tamsulosin) by weight of the microsphere.

In some embodiments, the microsphere may include from about 1% (w/w) to about 30% (w/w) (e.g., 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% (w/w)) of the active agent (e.g., oxybutynin, tolterodine, solifenacin, darifenacin, pramipexole, or tamsulosin) by weight of the microsphere.

“Isomers” are different compounds that have the same molecular formula. “Stereoisomers” are isomers that differ only in the way the atoms are arranged in space, i.e., having a different stereochemical configuration. “Enantiomers” are a pair of stereoisomers that are non-superimposable mirror images of each other. A 1:1 mixture of a pair of enantiomers is a “racemic” mixture. The term “(+)” is used to designate a racemic mixture where appropriate. “Diastereoisomers” are stereoisomers with at least two asymmetric atoms, but which are not mirror-images of each other. The absolute stereochemistry is specified according to the Cahn-Ingold-Prelog R-S system. When a compound is a pure enantiomer, the stereochemistry at each chiral carbon can be specified by either (R) or (S). Resolved compounds whose absolute configuration is unknown can be designated (+) or (−) depending on the direction (dextro- or levorotatory) in which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions, and methods are meant to include all such possible isomers, including racemic mixtures, optically pure forms, and intermediate mixtures. Optically active (R)- and (S)-isomers can be prepared using chiral synthons or chiral reagents or resolved using conventional techniques. When the compounds described herein contain olefinic double bonds or other centers of geometric asymmetry, and unless specified otherwise, it is intended that the compounds include both E and Z geometric isomers.

Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related to mirror images, most commonly because they contain two or more asymmetrically substituted carbon atoms. “R” and “S” represent the configuration of substituents around one or more chiral carbon atoms. Thus, “R*” and “S*” denote the relative configurations of substituents around one or more chiral carbon atoms. The symbol “*” in a structural formula represents the presence of a chiral carbon center.

“Racemate” or “racemic mixture” means a compound of equimolar quantities of two enantiomers, wherein such mixtures exhibit no optical activity, i.e., they do not rotate the plane of polarized light.

“Geometric isomer” means isomers that differ in the orientation of substituent atoms in relationship to a carbon-carbon double bond, a cycloalkyl ring, or a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “St,” “R*,” “E,” “Z,” “cis,” and “trans” indicate configurations relative to the core molecule.

A “derivative,” as used herein, refers to a chemical substance related structurally to another, i.e., an “original” substance, which can be referred to as a “parent” compound. A “derivative” can be made from the structurally related parent compound in one or more steps. The phrase “closely related derivative” means a derivative whose molecular weight does not exceed the weight of the parent compound by more than 50%. The general physical and chemical properties of a closely related derivative are also similar to the parent compound. “Pharmaceutically active derivative” refers to any compound that, upon administration to the recipient, can provide, directly or indirectly, the activity disclosed herein.

An “analog” refers to a small organic compound, a nucleotide, a protein, or a polypeptide that possesses similar or identical activity or function(s) as the compound, nucleotide, protein or polypeptide or compound having the desired activity of this disclosure, but need not necessarily include a sequence or structure that is similar or identical to the sequence or structure of the preferred embodiments.

A “prodrug” refers to a compound that may be converted under physiological conditions or by solvolysis to a biologically active compound described herein. Thus, the term “prodrug” refers to a precursor of a pharmaceutically acceptable and biologically active compound. A prodrug may be inactive when administered to a subject, but is converted in vivo to an active compound, for example, by hydrolysis. The prodrug compound often offers the advantages of solubility, tissue compatibility, or delayed release in a mammalian organism (see, e.g., Bundgaard, H., Design of Prodrugs (1985) (Elsevier, Amsterdam). The term “prodrug” also refers to any covalently bonded carriers, which release the active compound in vivo when administered to a subject. Prodrugs of an active compound, as described herein, may be prepared by modifying functional groups present in the active compound in such a way that the modifications are cleaved, either in routine manipulation or in vivo, to yield the active parent compound. Prodrugs include, for example, compounds wherein a hydroxy, amino or mercapto group is bonded to any group that, when the prodrug of the active compound is administered to a mammalian subject, cleaves to form a free hydroxy, free amino, or free mercapto group, respectively. Examples of prodrugs include, but are not limited to, acetates, formates, and benzoate derivatives of alcohol, various ester derivatives of a carboxylic acid, or acetamide, formamide, and benzamide derivatives of an amine functional group in the active compound. Various forms of prodrugs are well known in the art and are described in: (a) The Practice of Medicinal Chemistry, Camille G. Wermuth et al., Ch 31, (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds. Ch 5, pgs 113-191 (Harwood Academic Publishers, 1991); and (d) Hydrolysis in Drug and Prodrug Metabolism, Bernard Testa and Joachim M. Mayer, (Wiley-VCH, 2003).

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable salt” refers to a salt of the administered compounds prepared from pharmaceutically acceptable non-toxic acids, including inorganic acids, organic acids, solvates, hydrates, or clathrates thereof.

### Polymer Carriers

Polymers suitable for use in the context of this disclosure may have high water-absorbing ability and are generally biocompatible and non-toxic to tissues and cells. In some embodiments, the polymers may be selected from sodium acrylate polymers, acrylamide polymers, acrylamide derivative polymers or copolymers, sodium acrylate and vinyl alcohol copolymers, saponification products of copolymer of vinyl acetate and acrylic acid ester, vinyl acetate and acrylic acid ester copolymer, vinyl acetate and methyl maleate copolymer, isobutylene-maleic anhydride crosslinked copolymer, starch-acrylonitrile graft copolymer and its saponification products, crosslinked sodium polyacrylate polymer, and crosslinked polyethylene oxide.

In some embodiments, the polymer carrier may include poly(lactide-co-glycolide) (PLGA), polylactide (PLA), polyglycolic acid (PLG), polyethylene glycol-PLA, PLA-polycaprolactone (PCL), a polyorthoester, a polyphosphazene, a polyphosphoester, or a combination thereof.

In some embodiments, the polymer carrier may include PLGA, PLA, or a combination thereof. In some embodiments, the polymer carrier is PLGA.

In some embodiments, the polymer carrier has a degradation half-life of at least 2 months under physiological conditions. As used herein, the term “physiological conditions” refers to the temperature, pH, osmotic pressure, ionic strength, viscosity, etc., compatible with live bacteria and/or viable in cultured yeast or mammalian cells.

### Pharmaceutical Compositions and Kits

In another aspect, this disclosure provides a composition that may include the microsphere described herein.

As used herein, the term “composition” or “pharmaceutical composition” refers to a mixture of at least one component useful within the disclosure with other components, such as carriers, stabilizers, diluents, dispersing agents, suspending agents, thickening agents, and/or excipients. The pharmaceutical composition facilitates administration of one or more components of the invention to an organism.

In some embodiments, the composition (e.g., injectable composition) as disclosed may include the microspheres in an amount from about 5% to about 90% (e.g., about 5%, 8%, 11%, 14%, 17%, 20%, 23%, 26%, 29%, 32%, 35%, 38%, 41%, 44%, 47%, 50%, 53%, 56%, 59%, 62%, 65%, 68%, 71%, 74%, 77%, 80%, 83%, 86%, 89%, 90%) by weight or the polymer carrier in an amount from about 10% to about 90% (e.g., about 5%, 8%, 11%, 14%, 17%, 20%, 23%, 26%, 29%, 32%, 35%, 38%, 41%, 44%, 47%, 50%, 53%, 56%, 59%, 62%, 65%, 68%, 71%, 74%, 77%, 80%, 83%, 86%, 89%, 90%) by weight. The relative amount of the microspheres and the carrier may change according to the need of a specific use scenario, depending on factors such as the size of the needle used, type of microspheres and carriers used, area of injection, and whether cells are associated with the microspheres prior to injection.

In some embodiments, the microsphere may contain a polymer carrier in the amount ranges from about 10% to about 50% (e.g., about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%) by weight for and from 50% to 90% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%) of the microsphere.

In some embodiments, the composition may include at least one anti-cryogenic or anti-freeze agent. In some embodiments, anti-cryogenic or anti-freeze agents may include a polyhydric alcohol constituting the polyol ester including diols (ethylene glycol, 1,3-propanediol, propylene glycol, 1,4-butanediol, 1,2-butanediol, 2-methyl-1,3-Propanediol, 1,5-pentanediol, neopentyl glycol, 1,6-hexanediol, 2-ethyl-2-methyl-1,3-propanediol, 1,7-heptanediol, 2-methyl-2-Propyl-1,3-propanediol, 2,2-diethyl-1,3-propanediol, 1,8-octanediol, 1,9-nonanediol, 1,10-decanediol, 1,11-undecanediol, 1,12-dodecanediol, etc.), polyol having 3 to 20 hydroxyl groups (trimethylol ether), Trimethylolpropane, trimethylolbutane, di-(trimethylolpropane), tri-(trimethylolpropane), pentaerythritol, di-(pentaerythritol), tri-(pentaerythritol), glycerol, polyglycerol (of glycerol) 2-trimer), 1,3,5-pentanetriol, sorbitol, sorbitan, sorbitol glycerin condensate, polyhydric alcohol such as adonitol, arabitol, xylitol, mannitol, xylose, arabinose, ribose, rhamnose, glucose, fructose, sugars such as galactose, mannose, sorbose, cellobiose, maltose, isomaltose, trehalose, sucrose, raffinose, gentianose, merentoose, and the like.

In some embodiments, the composition may include an additional therapeutic agent. In some embodiments, the additional therapeutic agent may include an α2δ subunit calcium channel modulator, a β3 adrenergic agonist, a spasmolytic, a neurokinin receptor antagonist, a bradykinin receptor antagonist, a nitric oxide donor, or a combination thereof.

Techniques and formulations generally may be found in Remmington's Pharmaceutical Sciences, Meade Publishing Co., Easton, PA. For systemic administration, injection is preferred, including intramuscular, intravenous, intraperitoneal, and subcutaneous. For injection, the agents can be formulated in liquid solutions, e.g., in physiologically compatible buffers such as Hank's solution or Ringer's solution. In addition, the agents may be formulated in solid form and redissolved or suspended immediately prior to use. Lyophilized forms are also included.

An aqueous suspension can be prepared, for example, by admixing the disclosed microspheres and/or at least one pharmaceutically acceptable salt thereof with at least one excipient suitable for manufacturing an aqueous suspension. Non-limiting examples of excipients suitable for the manufacture of an aqueous suspension may include suspending agents, such as, for example, sodium carboxymethylcellulose, methylcellulose, hydroxypropylmethylcellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long-chain aliphatic alcohols, such as, for example, heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers, including saline, dextrose, or water, or with cyclodextrin (i.e., Captisol), cosolvent solubilization (i.e., propylene glycol) or micellar solubilization (i.e., Tween 80).

The sterile injectable preparation may also be a sterile injectable solution or suspension in a non-toxic, parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid can be used in the preparation of injectables.

Pharmaceutically acceptable carriers, adjuvants, and vehicles that may be used in the pharmaceutical compositions of this disclosure include, but are not limited to, ion exchangers, alumina, aluminum stearate, lecithin, self-emulsifying drug delivery systems (SEDDS) such as alpha-tocopherol polyethylene glycol 1000 succinate, surfactants used in pharmaceutical dosage forms such as Tweens, polyethoxylated castor oil, such as cremophor surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkyl cyclodextrins, including 2- and 3-hydroxypropyl-cyclodextrins, or other solubilized derivatives may also be advantageously used to enhance delivery of compounds of the formulae described herein.

The pharmaceutically active compounds of this disclosure can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as additives, preservatives, stabilizers, wetting agents, emulsifiers, buffers etc. Tablets and pills can additionally be prepared with enteric coatings. Such compositions may also include adjuvants, such as wetting, sweetening, flavoring, and perfuming agents.

Pharmaceutical compositions of this disclosure may include at least one compound and/or at least one pharmaceutically acceptable salt thereof, and optionally an additional agent selected from any pharmaceutically acceptable carrier, adjuvant, and vehicle. Alternate compositions of this disclosure may include a compound described herein, or a prodrug thereof, and a pharmaceutically acceptable carrier, adjuvant, or vehicle.

The microspheres or the pharmaceutical composition thereof described herein can be provided in a kit. In some embodiments, the kit includes a container that contains the microsphere, or the composition thereof, and optionally informational material. The informational material can be descriptive, instructional, marketing, or other material related to the methods described herein and/or the use of the agents for therapeutic benefit. For example, kits may include instructions for the manufacturing, the therapeutic regimen to be used, and periods of administration. In some embodiments, the kit may also include an additional therapeutic agent. The kit may include one or more containers, each with a different reagent. For example, the kit may include a first container that contains the composition and a second container for the additional therapeutic agent.

The containers may include a unit dosage of the pharmaceutical composition. In addition to the composition, the kit can include other ingredients, such as a solvent or buffer, an adjuvant, a stabilizer, or a preservative. The kit may optionally include a device suitable for administration of the composition, e.g., a syringe or other suitable delivery device. The device may be provided pre-loaded with one or both of the agents or can be empty, but suitable for loading.

Also provided in this disclosure is an implant comprising the microsphere or the composition thereof, as described herein. In some embodiments, the implant is a medical implant. As used herein, the term “medical implant” refers to an article that is designed to be placed at a target location in the body and reside at that target location for a period of time. The medical implants may also be bioactive agent releasing and biodegradable. In some embodiments, the medical implants may be fabricated having a high drug loading capacity, but were still able to release the bioactive agent (e.g., microspheres) at a steady, therapeutically effective rate. This allows the implants to be useful for the prolonged release of bioactive agents to treat medical conditions. For example, in some cases the implants can be formed to release the bioactive agent in a therapeutically useful amount for a period of time greater than one month, three months, six months, or even a year. Given the prolonged release of bioactive agent, the need for periodic administration of the bioactive agent is not required. This is beneficial as it eliminates or significantly reduces need for patient compliance.

### Methods of Preparing Microsphere-Based Formulations

In yet another aspect, this disclosure also provides a method of preparing a microsphere described herein. In some embodiments, the method may employ single/double emulsion extraction techniques for preparation of active agent(s) loaded microspheres. Notably, the release profile of an active agent (e.g., oxybutynin, tolterodine, solifenacin, darifenacin, pramipexole, or tamsulosin) from microspheres can be controlled by various factors, such as concentration of the polymer carrier solution (e.g., PLGA), polymer-drug solution, the temperature of both oil and aqueous phases, content and pH of aqueous phase, content and pH of dispersed phase, and content and pH of the hardening solution for solvent extraction, an agitation rate, or “quenching” the powdery lyophilizates applied during the preparation of the active agent loaded microspheres.

As used herein, the term “release profiles” refers to the dissolution profile of formulations as established by the in vitro studies. More particularly, the dissolution profile refers to the amount of the active agent that is released from the microspheres as a function of time in PBS (0.05 M, 7.4, 0.01% NaN₃) at 37° C. with 75 rpm.

In some embodiments, the release profiles of microspheres can be characterized by nearly zero-order kinetics (e.g., with a low initial burst on the first day, and then with constant release) and a R² of about 0.997. In some embodiments, the microspheres release 98-100% of the active agent (e.g., oxybutynin, tolterodine, solifenacin, darifenacin, pramipexole, or tamsulosin) over a controlled period of time.

In some embodiments, an example process for preparing an active agent (e.g., oxybutynin, tolterodine, solifenacin, darifenacin, pramipexole, or tamsulosin) loaded microspheres (e.g., PLGA microspheres) may include:
(1) dissolving at least one polymer (e.g., PLGA, PLA, etc.) in an organic solvent non-miscible with water to make a polymer solution.
(2) dissolving the active agent in the polymer solution to obtain a dispersed oil phase (or oxybutynin HCL in an aqueous solution.
(3) preparing an oil-in-water (O/W) or water in oil in water water-in-oil-in-water (W/O/W) emulsions, the oil phase comprised of an organic solvent non-miscible with water or aqueous solution contained in W/O/W and polymers; and
(4) extracting the organic solvent used in step (1) from the emulsion to form microspheres by roughly controlling the temperature during the extraction step. The hardening solution's temperature gradually increases under stages of the ice-cooled water bath and the ambient environment without heating.

In some embodiments, the concentrations of the polymer solution can range from 50 mg/ml to 1200 mg/ml, 300 mg/ml to 1000 mg/ml, 500 mg/ml to 850 mg/ml, 650 mg/ml to 800 mg/ml, or 700 mg/ml. In some embodiments, the agitation rate can be from 3000 rpm to 15000 rpm, 4500 rpm to 10000 rpm, or any intermediate range therebetween.

Also, both the frequency and duration of quenching the powdery lyophilizates greatly influence the drug release profiles. In some embodiments, the powdery lyophilizates can be quenched once, twice, 3, 4, 5 times or more. In some embodiments, the powdery lyophilizates can be quenched 1 to 3 times.

In some embodiments, the duration of quenching can be minutes, days, weeks, and months. In some embodiments, the duration of quenching can be days to weeks.

In some embodiments, the method employing the single emulsion/solvent extraction technique may include:
(1) dissolving PLGA in dichloromethane (DCM) to obtain a polymer solution.
(2) dissolving an active agent (e.g., oxybutynin) free base in the polymer solution made in the previous step and pre-cooled to about 45° F. to obtain a dispersed, oil phase.
(3) pre-cooling a 1% PVA solution containing PBS (0.01 M, pH adjusted to 8.1) to about 45° F. to obtain an aqueous/continuous phase.
(4) adding the dispersed, oil phase obtained in step (2) into the continuous phase made in step (3) while agitating at a predetermined rate to form the oil-in-water (O/W) emulsion (semi-solid microsphere suspension) of a desired particle size distribution.
(5) while stirring at a predetermined rate, transferring the O/W emulsion into a suitable container with a 10 times greater volume of water than that of the O/W emulsion for four hours, where the water as a hardening solution is pre-cooled to about 45° F., and the container is placed in an ice-cooled water bath of about 45° F. During the first three hours, the temperature of the hardening solution rises from about 45° F. to about 55° F. Then, the ice bath is removed, and the container is left in an ambient environment without heating equipment for another hour to complete the solvent extraction step. After this four-hour step, the temperature of the hardened microsphere suspension reaches about 66° F.;
(6) screening the hardened microspheres by a sieve (e.g., a #150 mesh sieve), and washing the harden microspheres with water through a filter (e.g., a 10 μm filter);
(7) re-suspending the harvested microspheres in quantum suficit (q.s.) mannitol solution for freeze drying to obtain a powdery lyophilizate; and
(8) quenching the obtained powdery lyophilizate.

In some embodiments, the temperature differences between samples and the environment used for “quenching” the powdery lyophilizates can be >10° F. In some embodiments, a −4° F. freezer may be used for quenching powdery lyophilizates. In some embodiments, powdery lyophilizates may be sealed in a closed container, and then transferred from room temperature into a −20° C. freezer for re-quenching to obtain the final product. In some embodiments, the quenching process may be repeated one or more times as needed and as a result, the quenching process may last an extended period of time (i.e., minutes, days, months, and years).

In some embodiments, the method employing the double emulsion extraction technique may include:
(i) dissolving PLGA in dichloromethane (DCM) to obtain a polymer solution.
(ii) dissolving the active agent in a gelatin solution to obtain the first water phase.
(iii) adding the polymer solution into the first water phase and emulsifying the mixture using sonication to obtain the primary emulsion (W/O), and then pre-cooling it down to about 45° F. to obtain an oil phase.
(iv) pre-cooling a 1% PVA solution containing 0.01 M PBS, pH 8.1 to about 45° F. to obtain a continuous phase.
(v) adding the oil phase obtained in step (iii) into the continuous phase made in step (iv) while agitating at a predetermined rate to form the W/O/W emulsion (the semi-solid microsphere suspension) of the desired particle size distribution.
(vi) while stirring at a predetermined rate, transferring the W/O/W emulsion into a suitable container with a 10 times greater volume of water than that of the W/O/W emulsion for four hours, where the water as a hardening solution is pre-cooled to about 45° F., and the container is placed in an ice-cooled water bath of about 45° F. During the first three hours, the temperature of the hardening solution rises from about 45° F. to about 55° F. Then, the ice bath is removed, and the container is left in an ambient environment without heating equipment for another hour to complete the solvent extraction step. After the four-hour step, the temperature of the hardened microsphere suspension reaches about 66° F.
(vii) screening the hardened microspheres by a sieve (e.g., a #150 mesh sieve), and washing the hardened microspheres with water through a filter (e.g., a 10 μm filter);
(viii) re-suspending the harvested microspheres in q.s. mannitol solution for freeze drying to obtain a powdery lyophilizate; and
(ix) transferring the obtained powdery lyophilizate from a room temperature to −20° C.

In some embodiments, emulsification of the dispersed oil phase in an aqueous, continuous phase is conducted by homogenization at 3,000-10,000 rpm to form a semi-solid microsphere suspension (O/W emulsion) of 1-200 μm, e.g., from 10 μm to 75 μm. The volume ratio of the oil phase to the aqueous phase (continuous phase) is 1:2 to 1:20.

In some embodiments, the aqueous phase for the solvent extraction step as a hardening solution can be a water-containing buffer (0.00 to 2 M, pH 1.0-14.0), e.g., pure water with pH 6.4-7.0. The ratio of the resulting O/W emulsion (semi-solid microsphere suspension) to the hardening solution in the solvent extraction step is 1:10 to 1:60, where pure water can be used as the hardening solution.

In some embodiments, the pure water used as the hardening solution for the solvent extraction step can be pre-cooled to about 32° F. In some embodiments, the hardening solution is pre-cooled to about 45° F.

In some embodiments, in the solvent extraction step, the above-formed O/W emulsion may be immediately transferred into a suitable container with water stirred at 170 rpm for 4 hours to extract the solvent from the semi-solid microspheres. The water in the container is pre-cooled to about 45° F., with a volume about 10 times greater than the W/O emulsion. During the first 3 hours, the container is immersed in the ice-cooled water bath at ambient temperature (e.g., about 71° F.). In the first 3 hours, the temperature of the hardening solution rises from about 45° F. to about 55° F., at which point the ice bath is removed, and the container remains at an ambient temperature without heating equipment for another hour to complete the whole solvent extraction step. After the 4-hour step, the temperature of the hardened microsphere suspension reaches about 66° F.

In some embodiments, the extraction of the solvent from the semi-solid microspheres is performed as soon as the semi-solid microspheres are transferred into a large volume of water with stirring.

In some embodiments, after the solvent extraction, the hardened microspheres are harvested by screening with a sieve (e.g., a #150 mesh sieve), washed using water through a filter (e.g., a filter with about 10 μm opening), and then re-suspended in q.s. mannitol solution for freeze drying.

In some embodiments, the method may include:
(a) dissolving a polymer carrier in at least one organic solvent that is substantially non-miscible with water to form a polymer solution.
(b) dissolving an active agent (e.g., oxybutynin, tolterodine, solifenacin, darifenacin, pramipexole, or tamsulosin) in the polymer solution by single emulsion/solvent extraction to obtain an oil phase drug-polymer solution, or by double emulsion to obtain a water-in-oil (W/O) primary emulsion, and cooling the oil phase drug-polymer solution or the water-in-oil primary emulsion to about 45° F.;
(c) adding the oil phase drug-polymer solution or the water-in-oil primary emulsion to a PVA solution, wherein the PVA solution is in the form of an aqueous, continuous phase.
(d) homogenizing the oil phase drug-polymer solution or the water-in-oil primary emulsion with the 1% PVA solution at a predetermined mixing rate to obtain a microsphere suspension having semi-solid microencapsulated droplets, wherein the microsphere suspension is in the form of oil-in-water or water-in-oil-in water (W/O/W) emulsion.
(e) extracting the organic solvent by:
placing a container containing water precooled to about 45° F. in an ice-cooled water bath,
transferring the microsphere suspension into the container while stirring the microsphere suspension with a mixer,
maintaining the container in the ice-cooled water bath for about 3 hours to allow the microsphere suspension to harden and the temperature thereof to rise from about 45° F. to about 55° F., and
removing the ice-cooled water bath and exposing the microsphere suspension to an ambient temperature without heating equipment for about 1 hour, such that the temperature of the hardened microsphere suspension reaches about 66° F.; and
(f) collecting microspheres by screening through a mesh sieve, washing with water through a filter, and re-suspending in a solution (e.g., a D-mannitol solution) for freeze-drying to obtain powdery lyophilized microspheres.

In some embodiments, the method further comprises: after step (f), quenching the powdery lyophilized microspheres one or more times at a temperature below 25 degrees Celsius. In some embodiments, the step of quenching is performed by placing the powdery lyophilized microspheres at −20 degrees Celsius.

In some embodiments, the polymer solution may contain from about 50 mg/mL to about 1200 mg/mL (e.g., 50, 100, 150, 200, 250, 300, 350, 400, 450, 500, 550, 600, 650, 700, 750, 800, 850, 900, 950, 1000, 1050, 1100, 1150, 1200 mg/mL) of the polymer carrier.

In some embodiments, the polymer carrier may include PLGA, PLA, PLG, polyethylene glycol-PLA, PCL, a polyorthoester, a polyphosphazene, a polyphosphoester, or a combination thereof.

In some embodiments, the polymer carrier may include PLGA. PLGA may be a commercially available PLGA, such as one available from Resomer® by Evonik Industries AG, Expansorb® by PC AS, PURASORB® by PURAC, Corbion. The PLGA polymers may have a molar ratio of lactic acid (LA) to glycolic acid (GA) in the range of about 0:100, 50:50, 75:25, and 100:0; the inherent viscosity (IV) in the range of 0.08-1.2 dl/g and their blends. In some embodiments, the PLGA used in the present disclosure may have an LA/GA monomer ratio of 75/25, and the inherent viscosity may be in the range of 0.16-0.2 dl/g.

In some embodiments, the organic solvent used in a polymer solution can be tetrahydrofuran, chloroform, dichloromethane (methylene chloride or DCM), acetonitrile, acetone, ethyl acetate, hexafluoro-isopropanol, and acetic acid. In some embodiments, the organic solvent is DCM.

In some embodiments, the polymer concentration in the organic solution may range from 50 mg/mL to 1,200 mg/mL, 200 mg/mL to 1,000 mg/ml, 300 to 800 mg/mL, 500-800 mg/ml, or 700-800 mg/ml.

In some embodiments, the aqueous, continuous phase may be an aqueous solution with one or more surfactants such as anionic surfactants, non-ionic surfactants (e.g., polyvinyl alcohol (PVA), poloxamers, Tweens), polyvinyl pyrrolidone, carboxymethylcellulose sodium, and gelatin are used alone or in a combination thereof.

In some embodiments, PVA may have an average molecular weight of from about 7000-23000 Da (e.g., 146,000-186,000 Da), 87-89% hydrolyzed.

In some embodiments, the PVA solution contains from about 0.1% (w/v) to about 5% (w/v) of PVA. In some embodiments, the PVA solution contains about 1% (w/v) of PVA.

In some embodiments, the PVA solution contains a buffer having a concentration of from about 0.0001 M to about 1 M. In some embodiments, the buffer has a concentration of about 0.05 M.

In some embodiments, the buffer has a pH of about 1 to about 14. In some embodiments, the buffer has a pH of about 8.1. In some embodiments, the buffer is a PBS buffer.

In some embodiments, the PVA solution at step (c) has a temperature of from about 35° F. to about 73° F. In some embodiments, the PVA solution at step (c) has a temperature of about 45° F.

In some embodiments, the predetermined mixing rate is from about 2000 rpm to about 20000 rpm.

In some embodiments, an aqueous phase in step (e) has a volume at least 2 times greater than an oil-in-water phase.

In some embodiments, the aqueous phase in step (e) may include a buffer with a concentration of from about 0.001 M to about 2 M. In some embodiments, the buffer has a pH of from about 1 to about 14.

In some embodiments, during step (e), the temperature of the microsphere suspension rises from about 35° F. to the ambient temperature. In some embodiments, the ambient temperature is about 73° F.

In some embodiments, when the container is in the ice-cooled water bath, the temperature of the microsphere suspension gradually increases as that of the ice-cooled water bath does. When the iced-cooled water bath is removed, the temperature of the microsphere suspension continues to increase toward the ambient temperature gradually.

In some embodiments, the active agent (e.g., oxybutynin, tolterodine, solifenacin, darifenacin, pramipexole, or tamsulosin) may include a free base, a stereoisomer, a derivative, an analog, a prodrug, or a pharmaceutically acceptable salt of the active agent.

In some embodiments, the drug-polymer solution (i.e., the dispersed, oil phase) contains the active agent with a concentration ranging from about 57 mg/mL to 1,340 mg/ml, about 230-1170 mg/ml, about 340 mg-910 mg/mL, or about 790-910 mg/ml.

In some embodiments, the active agent is dissolved in the polymer solution in the oil phase preparation using sonication to facilitate dissolving and eliminating air from the drug-polymer solution. The solution is then pre-cooled to about 45° F.

To prepare an injectable suspension of the disclosed microspheres, dried, sterilized microspheres may be mixed with a solvent for a pre-determined time to control the pre-injection swelling of the microspheres. The solvent can be pre-sterilized, or the suspension of microspheres, and the solvent can be sterilized together before injection thereof. Factors such as the material, size, and crosslinking degree of the microspheres; the type, volume, salt concentration, pH level, and temperature of the solvent; and the mixing time are considered before an injectable suspension is made, and the injection is carried out thereafter.

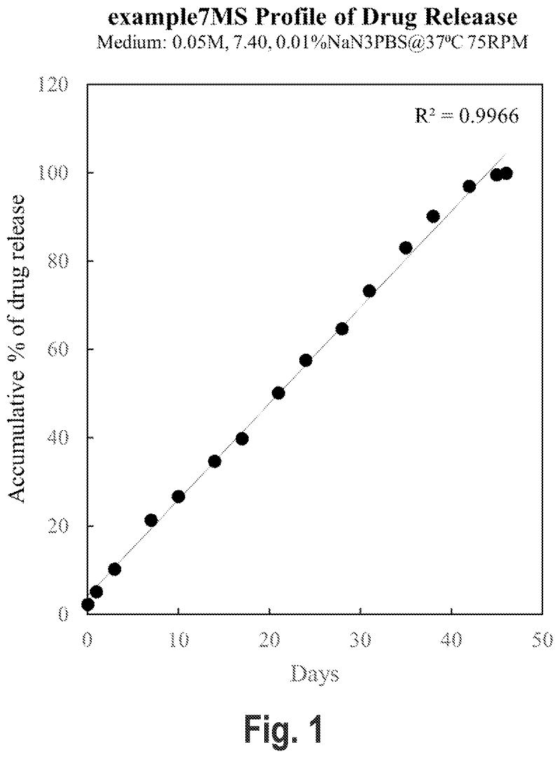

The microspheres or the compositions thereof can be readily injectable, e.g., through needles of 18 to 26 gauge, such as 22 to 24 gauge, into all parts of the mammal in need of treatment without causing significant pain or discomfort. This is due to, among other factors, the size and the physical resiliency of the microspheres, the biocompatible nature of the carrier, and the amount of the composition administered in accordance with the character and location of the tissue defects. The microspheres or the composition thereof as prepared according to the disclosed methods may be characterized by various suitable methods. Example methods for measuring particle size of microspheres, stability, drug loading efficiency, and in vitro drug release are provided below. For example, the results are summarized in Tables 1 to 12 and FIGS. 1 to 3. Selected formulations were characterized (see, e.g., Examples 7, 21, and 23) in vitro (FIGS. 1 to 3).

### Particle Size Measurement

The particle size of microspheres can be observed and determined by a microscope. In some embodiments, particle size can be measured by laser diffraction using a Malvern Master Sizer 2000Hydro 2000s. Average particle size can be expressed as the volume mean diameter in microns.

### Determination of Drug Loading Efficiency

About 20 mg of microspheres may be added to 10 mL acetonitrile using the vortex for 20 seconds before and after a 20-minute interval. 1 mL methanol and 3 mL water may be added to the suspension to extract the active agent (e.g., oxybutynin, tolterodine, solifenacin, darifenacin, pramipexole, or tamsulosin) and then vortexed until the mixture becomes clear. Next, 1 mL of the mixture may be added to 5 mL water and mixed well. The samples may be filtered through a filter (e.g., a 0.45 μm PTFE syringe filter) and then analyzed by HPLC to measure the amount of the active agent.

The percentage of loading efficiency is calculated by the tested drug loading rate to the theoretical rate of the drug entrapment or calculated by [the amount of feeding API-the amount of API lost in hardening solution)/the amount of feeding API* 100%. API: active pharmaceutical ingredient. The Agilent HPLC conditions (referred to in the Analytic Information of oxybutynin from Harman Finochem Ltd.) are as follows:
Column: Zorbax Eclipse XDB-C8 4.6×75 mm (3.5 μm)
Mobile Phase consists of Phosphate Buffer and CAN=45:55.
Running time: 10 minutes
The flow rate: 1 mL/min
Injection vol. 10 μL and the detection wavelength: 210 nm.

### Stability Study

The API's amount in samples is determined at different time points.

About 20 mg of microspheres were added to 10 mL ACN using the vortex for 20 seconds before and after a 20-minute interval. 1 ml methanol and 3 ml water were added into the suspension to extract the active agent and then vortexed until the mixture became clear; 1 ml of this mixture was added to 5 ml water and mixed well. The samples were filtered through a 0.45 μm PTFE syringe filter and then analyzed by HPLC to measure the amount of the active agent.

The percentage of API content is calculated using the ratio of the tested API amount to the weight of the drug sample.

The Agilent HPLC conditions (referred to in the Analytic Information of oxybutynin from Harman Finochem Ltd) are as follows:
Column: Zorbax Eclipse XDB-C8 4.6×75 mm (3.5 μm)
Mobile Phase consists of Phosphate Buffer and ACN=45:55.
Running time: 10 minutes
The flow rate: 1 mL/min
Injection vol. 10 μL and the detection wavelength: 210 nm.

### Determination of In Vitro Release

About 30 mg of the formulated microspheres are weighed into a 45-mL tube filled with 30 mL PBS (0.05 M, pH 7.4, 0.01% NaN₃) and immersed into a reciprocating water bath with 75 rpm at 37° C. The release medium is replaced at the following time points: 2 hours, days 1, 3, 7, 10, 14, 17, 21, 24, 28, 31, 35, until release is completed and then filtered through a 0.45 μm PES syringe filter for HPLC analysis.

The Agilent HPLC conditions (referred to in the Analytic Information of oxybutynin from Harman Finochem Ltd.) are as follows:
Column: Zorbax Eclipse XDB-C8 4.6×75 mm (3.5 μm)
Mobile Phase consists of Phosphate Buffer and CAN=45:55.
Running time: 10 minutes
The flow rate: 1 mL/min
Injection vol. 10 μL and the detection wavelength: 210 nm.

### Methods of Treatment

In another aspect, this disclosure further provides a method of treating or preventing a lower urinary tract disorder or reducing muscle spasms of bladder and urinary tract in a subject in need thereof. In some embodiments, the method may include administering to the subject a therapeutically effective amount of the microsphere or the composition, as described herein.

In some embodiments, the lower urinary tract disorder is characterized by at least one symptom selected from the group consisting of urinary frequency, urinary urgency, and nocturia.

In yet another aspect, this disclosure also provides a method of reducing muscle spasms of bladder and urinary tract in a subject in need thereof. In some embodiments, the method may include administering to the subject an effective amount of the microsphere or the composition, as described herein.

As used herein, the terms “treating,” “treat,” and “treatment” include preventing a disease, pathologic or medical condition from occurring (e.g., prophylaxis); inhibiting the disease, pathologic or medical condition or arresting its development; relieving or ameliorating the disease, pathologic or medical condition; and/or diminishing symptoms associated with the disease, pathologic or medical condition. Thus, the terms “treat,” “treatment,” and “treating” can extend to prophylaxis and can include preventing, prevention, lowering, stopping, or reversing the progression or severity of the condition or symptoms being treated. As such, the term “treatment” can include medical, therapeutic, and/or prophylactic administration, as appropriate. The term “treating” or “treatment” thus can include reversing, reducing, or arresting the symptoms, clinical signs, and underlying pathology of a condition in a manner to improve or stabilize a subject's condition.

As used herein, the term “administering” refers to the delivery of cells by any route including, without limitation, oral, intranasal, intraocular, intravenous, intraosseous, intraperitoneal, intraspinal, intramuscular, intra-articular, intraventricular, intracranial, intralesional, intratracheal, intrathecal, subcutaneous, intradermal, transdermal, or transmucosal administration. In some embodiments, the agent is administered to the subject intratumorally, intravenously, subcutaneously, intraosseously, orally, transdermally, sublingually, in sustained release, in controlled release, in delayed release, or as a suppository.

In some embodiments, the microsphere or the composition is administered to the subject intramuscularly or subcutaneously.

In some embodiments, the microsphere or the composition is administered to the subject as part of an implant.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect. A “therapeutically effective amount” of a compound with respect to the subject method of treatment refers to an amount of the compound in a preparation which, when administered as part of a desired dosage regimen (to a mammal, e.g., a human), alleviates a symptom, ameliorates a condition, or slows the onset of disease conditions according to clinically acceptable standards for the disorder or condition to be treated, e.g., at a reasonable benefit/risk ratio applicable to any medical treatment.

The actual dosage amount of a composition of this disclosure administered to a subject can be determined by physical and physiological factors such as body weight, severity of condition, the type of disease being treated, previous or concurrent therapeutic interventions, idiopathy of the patient, and on the route of administration. The practitioner responsible for administration will, in any event, determine the concentration of active ingredient(s) in a composition and appropriate dose(s) for the individual subject.

In some embodiments, the microspheres or the composition thereof may be administered at one or more doses of from about 0.5 mg/kg (mpk) to about 5 mg/kg (mpk) of body weight of the subject, such as about 0.5 mpk of body weight, about 1 mpk of body weight, about 1.5 mpk of body weight, about 2 mpk of body weight, about 2.5 mpk of body weight, about 3 mpk of body weight, about 3.5 mpk of body weight, about 4 mpk of body weight, about 4.5 mpk of body weight, about 5 mpk of body weight, or any range derivable therein.

In some embodiments, a dose may be presented in a single dose or as divided doses administered at appropriate intervals, for example, as two, three, four or more sub-doses per day. The sub-dose itself may be further divided, e.g., into a number of discrete, loosely spaced administrations.

In some embodiments, one or more doses of the microspheres or the composition may be administered at least every 1 day, every 2 days, every 3 days, every 4 days, every 5 days, every 6 days, every 7 days, every 1 week, every 2 weeks, every 3 weeks, every 4 weeks, every 5 weeks, every 6 weeks, every 7 weeks, every 8 weeks, every 9 weeks, every 10 weeks, every 11 weeks, every 12 weeks, every 13 weeks, every 14 weeks, every 15 weeks, every 16 weeks, every 17 weeks, every 18 weeks, every 19 weeks, every 20 weeks, every 21 weeks, every 22 weeks, every 23 weeks, every 24 weeks, every 25 weeks, every 26 weeks, every 27 weeks, every or 28 weeks.

In some embodiments, the microsphere or the composition maintains a sustained release of the active agent (e.g., oxybutynin, tolterodine, solifenacin, darifenacin, pramipexole, or tamsulosin) from about 4 weeks to about 18 weeks (e.g., about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks) in the subject.

### Combination Therapies

In some embodiments, the method may further include administering to the subject a second therapeutic agent. In some embodiments, the method may include administering to the subject an effective amount of the microsphere or the composition, as described herein, in combination with a second therapeutic agent.

In some embodiments, the second therapeutic agent may include an α2δ subunit calcium channel modulator, a β3 adrenergic agonist, a spasmolytic, a neurokinin receptor antagonist, a bradykinin receptor antagonist, a nitric oxide donor, or a combination thereof. In some embodiments, the α2δ subunit calcium channel modulator may include gabapentin, pregabalin, or a combination thereof.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “co-administration” or “co-administered” refers to the administration of at least two agent(s) or therapies to a subject. In some embodiments, the co-administration of two or more agents/therapies is concurrent. In some embodiments, a first agent/therapy is administered prior to a second agent/therapy. Those of skill in the art understand that the formulations and/or routes of administration of the various agents/therapies used may vary.

It is also possible to combine an agent (e.g., microspheres) with one or more other active ingredients in a unitary dosage form for simultaneous or sequential administration to a patient. The combination therapy may be administered as a simultaneous or sequential regimen. When administered sequentially, the combination may be administered in two or more administrations.

The combination therapy may provide synergy and be synergistic, i.e., the effect achieved when the active ingredients used together are greater than the sum of the effects that result from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills, capsules, or by different injections in separate syringes. In general, during alternation therapy, an effective dosage of each active ingredient is administered sequentially, i.e., serially, whereas, in combination therapy, effective dosages of two or more active ingredients are administered together. A synergistic effect denotes an effect that is greater than the predicted purely additive effects of the individual compounds of the combination.

Combination therapy is further described by U.S. Pat. Nos. 11103514, 10702495, 9382215, and 6833373, which include additional active agents that can be combined with the compounds described herein, and additional types of ailments and other conditions that can be treated with a compound or combination of compounds described herein.

An active agent may precede or follow treatment of the other agent by intervals ranging from minutes to weeks. In embodiments where the other agent and expression construct are applied separately to a cell, one would generally ensure that a significant period of time did not elapse between the time of each delivery, such that the agent and expression construct would still be able to exert an advantageously combined effect on the cell. For example, in such instances, it is contemplated that one may contact the cell, tissue or organism with two, three, four or more modalities substantially simultaneously (i.e., within less than about a minute) with the disclosed active.

In some embodiments, one or more agents may be administered within about 1 minute, about 5 minutes, about 10 minutes, about 20 minutes, about 30 minutes, about 45 minutes, about 60 minutes, about 2 hours, about 3 hours, about 4 hours, about 6 hours, about 8 hours, about 9 hours, about 12 hours, about 15 hours, about 18 hours, about 21 hours, about 24 hours, about 28 hours, about 31 hours, about 35 hours, about 38 hours, about 42 hours, about 45 hours, to about 48 hours or more prior to and/or after administering the disclosed active agent. In certain other embodiments, an agent may be administered within from about 1 day, about 2 days, about 3 days, about 4 days, about 5 days, about 6 days, about 8 days, about 9 days, about 12 days, about 15 days, about 16 days, about 18 days, about 20 days, to about 21 days prior to and/or after administering the disclosed active. In some embodiments, it may be desirable to extend the period for treatment significantly; however, where several weeks (e.g., about 1, about 2, about 3, about 4, about 6, or about 8 weeks or more) lapse between the respective administrations.

Administration of the compositions to a patient will follow general protocols for the administration of therapeutics, taking into account the toxicity, if any. It is expected that the treatment cycles will be repeated as necessary. It also is contemplated that various standard therapies or adjunct therapies, as well as surgical intervention, may be applied in combination with the described active agent. These therapies include but are not limited to chemotherapy, radiotherapy, immunotherapy, gene therapy, and surgery.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, a “subject” or “subject in need thereof” refers to a human and a non-human animal. Examples of a non-human animals include all vertebrates, e.g., mammals, such as non-human mammals, non-human primates (particularly higher primates), dog, rodent (e.g., mouse or rat), guinea pig, cat, and rabbit, and non-mammals, such as birds, amphibians, reptiles, etc. In some embodiments, the subject is a human. In another embodiment, the subject is an experimental animal or animal suitable as a disease model.

The term “disease” as used herein is intended to be generally synonymous, and is used interchangeably with the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder, or pathological condition.

Doses are often expressed in relation to body weight. Thus, a dose which is expressed as [g, mg, or other unit]/kg (or g, mg, etc.) usually refers to [g, mg, or other unit] “per kg (or g, mg, etc.) bodyweight,” even if the term “bodyweight” is not explicitly mentioned.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “In some embodiments,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges are meant to be encompassed within the scope of the present invention. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. When used in this document, the term “exemplary” means “by way of example” and does not indicate that a particular exemplary item is preferred or required.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

The following examples of parenteral injectable oxybutynin microspheres demonstrate the present invention in detail and are in no way meant to limit the scope thereof.

Materials and reagents used in the examples were purchased from commercial sources. PLGA and PLA polymers were obtained from PURAC, Corbion; Oxybutynin free base was obtained from Harman Finochem Ltd, Aurangabad, India; D-mannitol was obtained from TCI AMERICA, Portland, OR, U.S.A; and dichloromethane (DCM) was obtained from Spectrum Chemical Mfg. Corp., New Brunswick, New Jersey, U.S.A. Pramipexole-free base was obtained from Harman Finochem Ltd, Aurangabad, India; Pramipexole Dihydrochloride Monohydrate and D-mannitol were obtained from TCI AMERICA, Portland, OR, U.S.A; Poly(Vinyl Alcohol) was purchased from Millipore Sigma Burlington, MA, United States; Dichloride Methylene(DCM), and Gelatin (Type B, 250 Bloom) was obtained from Spectrum Chemical Mfg. Corp., New Brunswick, New Jersey, U.S.A.

Oxybutynin microspheres in Examples 1-18 were prepared by a single emulsion/solvent extraction method.

### Example 1

1070.4 mg of Poly(DL-lactide-co-glycolide) (PLGA) (75/25, 0.16 dl/g PURASORB® PDLG75016A) was dissolved in 1.5 mL of dichloromethane (DCM) with magnetic stirring to obtain a polymer solution with a concentration of 700 mg/ml. 144.7 mg of oxybutynin free base was dissolved in the above polymer solution, and the sonication was used to facilitate the process of oxybutynin dissolving and to eliminate air from the drug-polymer solution. The oxybutynin polymer solution was then pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 20 mL of the 1% PVA solution while homogenizing at 10,000 rpm for 45 seconds to form a semi-solid microencapsulated oil droplet (O/W emulsion) suspension. A container with 200 mL of water pre-cooled to 45° F. was placed in an ice-cooled water bath. Under stirring at 170 rpm, the O/W emulsion was transferred immediately into the container. The container was maintained in the ice-cooled water bath for 3 hours to complete the solvent extraction step. The hardened microspheres were collected by screening through a #150 mesh sieve, washed with water through a 10 μm opening filter, and re-suspended in 10 mL of mannitol solution containing 100 mg mannitol for freeze drying to obtain a powdery lyophilizate. The powdery lyophilizates were placed in a closed container at room temperature, and then stored at a −20° C. freezer. The drug loading efficiency obtained from the above process is about 94.46%.

### Example 2

1028.9 mg of PLGA (50/50, 0.2 dl/g PURASORB® 5002AY) was dissolved in 1.47 mL of DCM with magnetic stirring to obtain a polymer solution with a concentration of 700 mg/ml. 141.0 mg of oxybutynin was dissolved in the polymer solution, and sonication was used to facilitate the process of dissolving oxybutynin and to eliminate air from the drug-polymer solution. Then, the oxybutynin polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 20 mL of the 1% PVA solution, while homogenizing at 10000 rpm for 45 seconds to form a semi-solid microencapsulated oil droplets (O/W emulsion) suspension. A container with 200 mL of water pre-cooled to 45° F. was placed in an ice-cooled water bath. Under stirring at 170 rpm, the O/W emulsion was transferred immediately into the container. The container was maintained in the ice-cooled water bath for 3 hours to complete the solvent extraction step. The hardened microspheres were collected by screening through a #150 mesh sieve, washed using water through a 10 μm opening filter, and re-suspended in 10 mL of mannitol solution containing 109.2 mg mannitol for freeze drying to obtain a powdery lyophilizate. The powdery lyophilizates were placed in a closed container at room temperature, and then stored in a −20° C. freezer. The drug loading efficiency obtained from the above process is about 96.74%.

### Example 3

1005.2 mg of PLGA (50/50, 0.4 dl/g PURASORB® 5004A) was dissolved in 1.44 mL of DCM with magnetic stirring to obtain a polymer solution with a concentration of 700 mg/ml. 136.1 mg of oxybutynin was dissolved in the polymer solution, and sonication was used to facilitate the process of dissolving oxybutynin and to eliminate air from the drug-polymer solution. Then, the oxybutynin polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 20 mL of the 1% PVA solution while homogenizing at 10000 rpm for 45 seconds to form a semi-solid microencapsulated oil droplet (O/W emulsion) suspension. A container with 200 mL of water pre-cooled to 45° F. was placed in an ice-cooled water bath. Under stirring at 170 rpm, the O/W emulsion was transferred immediately into the container. The container was maintained in the ice-cooled water bath for 3 hours to complete the solvent extraction step. The hardened microspheres were collected by screening through a #150 mesh sieve, washed using water through a 10 μm opening filter, and re-suspended in 10 mL of mannitol solution containing 100.0 mg mannitol for freeze drying to obtain a powdery lyophilizate. The powdery lyophilizates were placed in a closed container at room temperature, and then stored in a −20° C. freezer. The drug loading efficiency obtained from the above process is about 99.10%.

### Example 4

1075.3 mg of PLGA (50/50, 0.2 dl/g PURASORB® 5002AY) and 272.5 mg of PLGA (75/25, 0.16 dl/g PURASORB® PDLG75016A) was dissolved in 1.92 mL of DCM with magnetic stirring to obtain a polymer solution with a concentration of 700 mg/ml. 188.7 mg of oxybutynin was dissolved in the above polymer solution, and sonication was used to facilitate the process of dissolving oxybutynin and to eliminate air from the drug-polymer solution. Then, the oxybutynin-polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 20 mL of the 1% PVA solution while homogenizing at 10000 rpm for 45 seconds to form a semi-solid microencapsulated oil droplets (O/W emulsion) suspension. A container with 200 mL of water pre-cooled to 45° F. was placed into an ice-cooled water bath. Under stirring at 170 rpm, the O/W emulsion was immediately transferred into the container. The container was maintained in the ice-cooled water bath for 3 hours to complete the solvent extraction step. The hardened microspheres were collected by screening through a #150 mesh sieve, washed using water through a 10 μm opening filter, and re-suspended in 10 mL of mannitol solution containing 200.0 mg mannitol for freeze-drying to obtain a powdery lyophilizate. The powdery lyophilizates were placed in a closed container at room temperature, and then stored at a −20° C. freezer. The drug loading efficiency obtained from the above process is about 94.28%.

### Example 5

269.8 mg of PLGA (50/50, 0.2 dl/g PURASORB® 5002Ay) and 1074.4 mg of PLGA (75/25, 0.16 dl/g PURASORB® PDLG75016A) was dissolved in 1.92 mL of DCM with magnetic stirring to obtain a polymer solution with a concentration of 700 mg/ml. 196.3 mg of oxybutynin was dissolved in the polymer solution, and sonication was used to facilitate the process of dissolving oxybutynin and eliminating air from the drug-polymer solution. The oxybutynin-polymer solution was then pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 20 mL of the 1% PVA solution while homogenizing at 10000 rpm for 45 seconds to form a semi-solid microencapsulated oil droplets (O/W emulsion) suspension. A container with 200 mL of water pre-cooled to 45° F. was placed in an ice-cooled water bath. Under stirring at 170 rpm, the O/W emulsion was transferred immediately into the container. The container was maintained in the ice-cooled water bath for 3 hours to complete the solvent extraction step. The hardened microspheres were collected by screening through a #150 mesh sieve, washed using water through a 10 μm opening filter, and re-suspended in 10 mL of mannitol solution containing 250.3 mg mannitol for freeze-drying to obtain a powdery lyophilizate. The powdery lyophilizates were placed in a closed container at room temperature, and then stored at a −20° C. freezer. The drug loading efficiency obtained from the process is about 92.52%

### Example 6

271.5 mg of PLGA (50/50, 0.2 dl/g PURASORB® 5002AY) and 1074.5 mg of PLGA (75/25, 0.2 dl/g PURASORB® PDLG7502A) was dissolved in 1.92 mL of DCM with magnetic stirring to obtain a polymer solution with a concentration of 700 mg/ml. 189.6 mg of oxybutynin was dissolved in the polymer solution, and sonication was used to facilitate the process of dissolving oxybutynin and eliminating air from the drug-polymer solution. Then, the oxybutynin-polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 20 mL of the 1% PVA solution while homogenizing at 10000 rpm for 45 seconds to form a semi-solid microencapsulated oil droplets (O/W emulsion) suspension. A container with 200 ml of water pre-cooled to 45° F. was placed in an ice-cooled water bath. Under stirring at 170 rpm, the O/W emulsion was transferred immediately into the container. The container was maintained in the ice-cooled water bath for 3 hours. The ice-cooled water bath was removed, and the container was exposed to the ambient environment for another hour to complete the solvent extraction step. The hardened microspheres were collected by screening through a #150 mesh sieve, washed using water through a 10 μm opening filter, and re-suspended in 20 mL of mannitol solution containing 261 mg mannitol for freeze-drying to obtain a powdery lyophilizate. The powdery lyophilizates were placed in a closed container at room temperature, and then stored at a −20° C. freezer. The drug loading efficiency obtained from the process is about 94.04%

### Example 7

1341.5 mg of PLGA (75/25, 0.2 dl/g PURASORB® PDLG7502A) was dissolved in 1.92 mL of DCM with magnetic stirring to obtain a polymer solution with a concentration of 700 mg/ml. 190.1 mg of oxybutynin was dissolved in the polymer solution, and sonication was used to facilitate the process of dissolving oxybutynin and eliminating air from the drug-polymer solution. Then, the oxybutynin-polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 20 mL of the 1% PVA solution while homogenizing at 9500 rpm for 45 seconds to form a semi-solid microencapsulated oil droplets (O/W emulsion) suspension. A container with 200 mL of water pre-cooled to 45° F. was placed in an ice-cooled water bath. Under stirring at 170 rpm, the O/W emulsion was transferred immediately into the container. The container was maintained in the ice-cooled water bath for 3 hours. The ice-cooled water bath was then removed, and the container was exposed to the ambient environment for another hour to complete the solvent extraction step. The hardened microspheres were collected by screening through a #150 mesh sieve, washed using water through a 10 μm opening filter, and re-suspended in 20 mL of mannitol solution containing 260.3 mg mannitol for freeze-drying to obtain a powdery lyophilizate. The powdery lyophilizates were placed in a closed container at room temperature, and then stored at a −20° C. freezer. The product yield from the above process is 73.05%, and the drug loading efficiency is about 94.40%.

All Examples 1-7 of oxybutynin-loaded microspheres were prepared with the previously mentioned formula and procedure except the ratio of lactic acid (LA) to glycolic acid (GA) or I.V of the PLGA. The sizes of the microspheres in all batches range from about 10 μm to about 75 μm. The in vitro release data for these samples are shown in Table 1.

In this group of examples, the formulation of Example 7 was selected as the optimal formulation due to size, size distribution, and shape of microspheres, % of encapsulation efficiency, % of drug release from microspheres, and the overall release characteristics of the formulation. The oxybutynin release characteristics of sample 7 in the in vitro study are shown in FIG. 1

TABLE 1
The drug release results of the in vitro oxybutynin microspheres
prepared at the different contents of PLGA.

| Time | Example | Example | Example | Example | Example | Example | Example |
|---|---|---|---|---|---|---|---|
| (days) | 1% | 2% | 3% | 4% | 5% | 6% | 7% |
| 0.083 | 4.0 | 1.8 | 1.5 | 4 | 3.3 | 2.2 | 2.3 |
| 1 | 10.0 | 6.0 | 5.9 | 10 | 7.6 | 5.9 | 5.1 |
| 3 | 18.2 | 12.2 | 12.4 | 18 | 14.6 | 11.5 | 10.3 |
| 7 | 31.4 | 18 | 23.3 | 25 | 25.4 | 21.2 | 21.3 |
| 10 | 36.0 | 23.3 | 28.2 | 30 | 30.5 | 28.8 | 26.6 |
| 14 | 41.8 | 33.0 | 37.2 | 43 | 40.7 | 37.0 | 34.7 |
| 17 | 48.5 | 38.5 | 42.3 | 51 | 47.8 | 43.2 | 39.8 |
| 21 | 57.4 | 42.7 | 49 | 62 | 57.1 | 52.8 | 50.1 |
| 24 | 55.1 | 44.5 | 53.6 | 69 | 64.8 | 59.8 | 57.4 |
| 28 | 77.4 | 47.0 | 59 | 75 | 70.3 | 64.1 | 64.6 |
| 31 | | 47.4 | 60.1 | 81 | 77.5 | 70.6 | 73.3 |
| 35 | | 49.6 | 62.3 | 88 | 88.5 | 77.4 | 83 |
| 38 | | | | 91 | 91.0 | 82.8 | 90.1 |
| 42 | | 51.9 | 65 | 94 | 95.6 | 87.8 | 96.9 |
| 45 | | | | 96 | 97.7 | 89.6 | 99.5 |
| 48 | | | | 97.5 | 97.9 | | 99.9 |
| | | | | | (d46) | | (d46) |
| 49 | | 53.5 | 66.5 | | | 91.3 | |
| 52 | | 54.3 | 67 | | | | |
| 54 | | 54.8 | 67.5 | | | | |
| 56 | | 55.3 | 68 | | | | |
| 59 | | 56.5 | 68.9 | | | | |
| 60 | | 56.6 | 69 | | | | |
| (forced | | 99.4 | 90.1 | 97.8 | 100.5 | 100.5 | 100 |
| release) | | (d60) | (d60) | (d48) | (d46) | (d49) | (d46) |
| recovered | | | | | | | |

### Example 8

1339 mg of PLGA (75/25, 0.2 dl/g PURASORB® PDLG7502A) was dissolved in 2.68 mL of DCM with magnetic stirring to obtain a polymer solution with a concentration of 500 mg/ml. 188.5 mg of oxybutynin was dissolved in the polymer solution, and sonication was used to facilitate the process of dissolving oxybutynin and eliminating air from the drug-polymer solution. Then, the oxybutynin-polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 30 mL of the 1% PVA solution while homogenizing at 9500 rpm for 45 seconds to form a semi-solid microencapsulated oil droplets (O/W emulsion) suspension. A container with 300 mL of water pre-cooled to 45° F. was placed in an ice-cooled water bath. Under stirring at 170 rpm, the O/W emulsion was transferred immediately into the container. The container was maintained in the ice-cooled water bath for 3 hours. The ice-cooled water bath was then removed, and the container was exposed to the ambient environment for another hour to complete the solvent extraction step. The hardened microspheres were collected by screening through a #150 mesh sieve, washed using water through a 10 μm opening filter, and re-suspended in 25 mL of mannitol solution containing 265 mg mannitol for freeze-drying to obtain a powdery lyophilizate. The powdery lyophilizates were placed in a closed container at room temperature, and then stored at a −20° C. freezer. The drug loading efficiency obtained from the process is about 89.54%.

### Example 9

1347 mg of PLGA (75/25, 0.2 dl/g PURASORB® PDLG7502A) was dissolved in 2.1 mL of DCM with magnetic stirring to obtain a polymer solution with a concentration of 650 mg/ml. 189.1 mg of oxybutynin was dissolved in the polymer solution, and sonication was used to facilitate the process of dissolving oxybutynin and to eliminate air from the drug-polymer solution. Then, the oxybutynin-polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 22 mL of the 1% PVA solution while homogenizing at 9400 rpm for 45 seconds to form a semi-solid microencapsulated oil droplets (O/W emulsion) suspension. A container with 220 mL of water pre-cooled to 45° F. was placed in an ice-cooled water bath. Under stirring at 170 rpm, the O/W emulsion was transferred immediately into the container. The container was maintained in the ice-cooled water bath for 3 hours. The ice-cooled water bath was then removed, and the container was exposed to the ambient environment for another hour to complete the solvent extraction step. The hardened microspheres were collected by screening through a #150 mesh sieve, washed using water through a 10 μm opening filter, and re-suspended in 25 mL of mannitol solution containing 261.3 mg mannitol for freeze-drying to obtain a powdery lyophilizate. The powdery lyophilizates were placed in a closed container at room temperature, and then stored at a −20° C. freezer. The drug loading efficiency obtained from the process is about 91.68%.

### Example 10

1344.6 mg of PLGA (75/25, 0.2 dl/g PURASORB® PDLG7502A) was dissolved in 1.68 mL of DCM with magnetic stirring to obtain a polymer solution with a concentration of 800 mg/ml. 190.3 mg of oxybutynin was dissolved in the polymer solution, and sonication was used to facilitate the process of dissolving oxybutynin and to eliminate air from the drug-polymer solution. Then, the oxybutynin-polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 20 mL of the 1% PVA solution while homogenizing at 9500 rpm for 45 seconds to form a semi-solid microencapsulated oil droplets (O/W emulsion) suspension. A container with 200 mL of water pre-cooled to 45° F. was placed in an ice-cooled water bath. Under stirring at 170 rpm, the O/W emulsion was transferred immediately into the container. The container was maintained in the ice-cooled water bath for 3 hours. The ice-cooled water bath was then removed, and the container was exposed to the ambient environment for another hour to complete the solvent extraction step. The hardened microspheres were collected by screening through a #150 mesh sieve, washed using water through a 10 μm opening filter, and re-suspended in 25 mL of mannitol solution containing 259.3 mg mannitol for freeze-drying to obtain a powdery lyophilizate. The powdery lyophilizates were placed in a closed container at room temperature, and then stored at a −20° C. freezer. The drug loading efficiency obtained from the above process is about 97.95%.

### Example 11

1342.9 mg of PLGA (75/25, 0.2 dl/g PURASORB® PDLG7502A) was dissolved in 1.79 mL of DCM with magnetic stirring to obtain a polymer solution with a concentration of 750 mg/ml. 191.3 mg of oxybutynin was dissolved in the polymer solution, and sonication was used to facilitate the process of dissolving oxybutynin and eliminating air from the drug-polymer solution. Then, the oxybutynin-polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 20 mL of the 1% PVA solution while homogenizing at 9400 rpm for 45 seconds to form a semi-solid microencapsulated oil droplets (O/W emulsion) suspension. A container with 200 mL of water pre-cooled to 45° F. was placed in an ice-cooled water bath. Under stirring at 170 rpm, the O/W emulsion was transferred immediately into the container. The container was maintained in the ice-cooled water bath for 3 hours. The ice-cooled water bath was then removed, and the container was exposed to the ambient environment for another hour to complete the solvent extraction step. The hardened microspheres were collected by screening through a #150 mesh sieve, washed using water through a 10 μm opening filter, and re-suspended in 25 mL of mannitol solution containing 258.9 mg mannitol for freeze-drying to obtain a powdery lyophilizate. The powdery lyophilizates were placed in a closed container at room temperature, and then stored at a −20° C. freezer. The drug loading efficiency obtained from the above process is about 94.67%.

### Example 12

1339 mg of PLGA (75/25, 0.2 dl/g PURASORB® PDLG7502A) was dissolved in 4.5 mL of DCM with magnetic stirring to obtain a polymer solution with a concentration of 300 mg/ml. 190.9 mg of oxybutynin was dissolved in the polymer solution, and sonication was used to facilitate the process of dissolving oxybutynin and to eliminate air from the drug-polymer solution. Then, the oxybutynin-polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 46 mL of the 1% PVA solution while homogenizing at 9400 rpm for 45 seconds to form a semi-solid microencapsulated oil droplets (O/W emulsion) suspension. Place a container with 450 mL of water pre-cooled to 45° F. into an ice-cooled water bath. Under stirring at 170 rpm, the O/W emulsion was transferred immediately into the container. The container was maintained in the ice-cooled water bath for 3 hours. The ice-cooled water bath was then removed, and the container was exposed to the ambient environment for another hour to complete the solvent extraction step. The hardened microspheres were collected by screening through a #150 mesh sieve, washed using water through a 10 μm opening filter, and re-suspended in 20 mL of mannitol solution containing 260.0 mg mannitol for freeze-drying to obtain a powdery lyophilizate. The powdery lyophilizates were placed in a closed container at room temperature, and then stored at a −20° C. freezer. The drug loading efficiency obtained from the above process is about 84.36%.

The group of Examples 7-12 of oxybutynin-loaded microspheres were prepared with the previously mentioned formula and procedure while varying the concentration of the PLGA solution. The sizes of the microspheres in all batches range from about 10 μm to about 75 μm. The in vitro release data for these examples are shown in Table 2.

In this group of examples, the formulation of Example 7 was selected as the optimal formulation due to size, size distribution and shape of microspheres, % of encapsulation efficiency, % of drug release from microspheres, and the overall release characteristics of the formulation. The oxybutynin release characteristics of Example 7 in the in vitro study are shown in FIG. 1

TABLE 2
The drug release results of the in vitro oxybutynin microspheres
prepared at the different concentrations of polymer solutions.

| | Concentration of polymer solution | | | | | |
|---|---|---|---|---|---|---|
| | 300 mg/ml | 500 mg/ml | 650 mg/ml | 700 mg/ml | 750 mg/ml | 800 mg/ml |
| Time(days) | Sample12% | Sample8% | Sample9% | Sample7% | Sample11% | Sample10% |
| 0.083 | 4.0 | 2.1 | 2.1 | 2.3 | 2.1 | 2.2 |
| 1 | 8.9 | 6.4 | 5.7 | 5.1 | 5.6 | 5.4 |
| 3 | 14.7 | 12 | 12 | 10.3 | 11.8 | 10.5 |
| 7 | 24.4 | 23.5 | 23.4 | 21.3 | 22.4 | 21.8 |
| 10 | 32.3 | 34.7 | 31.5 | 26.6 | 30.0 | 30.4 |
| 14 | 41.2 | 44.6 | 41.9 | 34.7 | 39.8 | 39.5 |
| 17 | 45.4 | 50.7 | 49.7 | 39.8 | 46.9 | 45.9 |
| 21 | 65.0 | 62.4 | 59.4 | 50.1 | 56.5 | 57.6 |
| 24 | 68.5 | 71.4 | 68.8 | 57.4 | 64.5 | 66.4 |
| 28 | 75.9 | 82.8 | 79 | 64.6 | 73.9 | 76.9 |
| 31 | 85.1 | 89.6 | 86.8 | 73.3 | 81.1 | 83.8 |
| 35 | 96.7 | 96.4 | 95.8 | 83 | 90.2 | 91.7 |
| 38 | 98.8(d37) | 98.9 | 99.5 | 90.1 | 97.3 | 96.2 |
| 42 | | 99.6 | 100 | 96.9 | 98.4 | 99.8 |
| 45 | | | | 99.5 | | |
| 46 | | | | 99.9 | | |
| (forced | 100.0(d37) | 100.0(d42) | 100.0(d42) | 100(d46) | 100.0(d42) | 100.0(d42) |
| release)recovered | | | | | | |

### Example 13

1339 mg of PLGA (75/25, 0.2 dl/g PURASORB® PDLG7502A) was dissolved in 4.5 mL of DCM with magnetic stirring to obtain a polymer solution with a concentration of 300 mg/ml. 190.9 mg of oxybutynin was dissolved in the polymer solution, and sonication was used to facilitate the process of dissolving oxybutynin and to eliminate air from the drug-polymer solution. Then, the oxybutynin-polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 46 mL of the 1% PVA solution while homogenizing at 7000 rpm for 45 seconds to form a semi-solid microencapsulated oil droplets (O/W emulsion) suspension. A container with 450 mL of water pre-cooled to 45° F. was placed in an ice-cooled water bath. Under stirring at 170 rpm, the O/W emulsion was transferred immediately into the container. The container was maintained in the ice-cooled water bath for 3 hours. The ice-cooled water bath was then removed, and the container was exposed to the ambient environment for another hour to complete the solvent extraction step. The hardened microspheres were collected by screening through a #150 mesh sieve, washed using water through a 10 μm opening filter, and re-suspended in 20 mL of mannitol solution containing 260.0 mg mannitol for freeze-drying to obtain a powdery lyophilizate. The powdery lyophilizates were placed in a closed container at room temperature, and then stored at a −20° C. freezer. The drug loading efficiency obtained from the above process is about 84.42%.

### Example 14

1340.5 mg of PLGA (75/25, 0.2 dl/g PURASORB® PDLG7502A) was dissolved in 4.5 mL of DCM with magnetic stirring to obtain a polymer solution with a concentration of 300 mg/ml. 191.8 mg of oxybutynin was dissolved in the polymer solution, and sonication was used to facilitate the process of dissolving oxybutynin and eliminating air from the drug-polymer solution. Then, the oxybutynin-polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 46 mL of the 1% PVA solution while homogenizing at 4500 rpm for 45 seconds to form a semi-solid microencapsulated oil droplets (O/W emulsion) suspension. A container with 450 mL of water pre-cooled to 45° F. was placed in an ice-cooled water bath. Under stirring at 170 rpm, the O/W emulsion was transferred immediately into the container. The container was maintained in the ice-cooled water bath for 3 hours. The ice-cooled water bath was then removed, and the container was exposed to the ambient environment for another hour to complete the solvent extraction step. The hardened microspheres were collected by screening through a #150 mesh sieve, washed using water through a 10 μm opening filter, and re-suspended in 20 mL of mannitol solution containing 260.0 mg mannitol for freeze-drying to obtain a powdery lyophilizate. The powdery lyophilizates were placed in a closed container at room temperature, and then stored at a −20° C. freezer. The drug loading efficiency obtained from the above process is about 84.37%.

Oxybutynin-loaded microspheres in Examples 12-14 were prepared with the previously mentioned formula and procedure while varying the agitation rate applied during the preparation of the O/W emulsion. The sizes of the microspheres in all batches range from about 10 μm to about 75 μm. The HPLC results indicate that the drug loading efficiency was not significantly influenced by the agitation rate applied during preparation of the W/O emulsion, but the in vitro release data for these examples show that the higher the agitation rate applied, the bigger initial burst of the first day and the earlier the drug release completed (Table 3).

TABLE 3
The drug encapsulation efficiency and the release
results of the in vitro oxybutynin microspheres prepared at the
different agitation rates for preparing the W/O emulsion.

| Samples | Example 12 | Example 13 | Example 14 |
|---|---|---|---|
| Agitation rate | 9,400 | 7,000 | 4,500 |
| (rpm) | | | |
| drug loading | 84.36 | 84.42 | 84.37 |
| efficiency (%) | | | |
| 2 hours | 4.0 | 3.4 | 1.9 |
| 1 day | 8.9 | 8.0 | 6.1 |
| 3 days | 14.7 | 13.1 | 10.8 |
| 7 | 24.4 | 22.4 | 19.4 |
| 10 | 32.3 | 29.8 | 27.0 |
| 14 | 41.2 | 37.0 | 35.4 |
| 17 | 45.4 | 40.7 | 40.0 |
| 21 | 65.0 | 50.9 | 49.5 |
| 24 | 68.5 | 60.3 | 58.7 |
| 28 | 75.9 | 71.6 | 71.3 |
| 31 | 85.1 | 81.7 | 80.1 |
| 35 | 96.7 | 93.3 | 90.3 |
| 38 | 98.8 | 98.6 | 96.4 |
| | (d37) | | |
| 42 | | 100.5 | 99.9 |
| 42 F | 100.0 | 100.7 | 100.4 |
| (recovered) | (d37F) | | |

### Example 15

1561.5 mg of PLGA (75/25, 0.2 dl/g PURASORB® PDLG7502A) was dissolved in 1.3 mL of DCM with magnetic stirring to obtain a polymer solution with a concentration of 1200 mg/ml. 190.3 mg of oxybutynin was dissolved in the polymer solution, and sonication was used to dissolve oxybutynin and eliminate air from the drug-polymer solution. Then, the oxybutynin-polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 20 mL of the 1% PVA solution while homogenizing at 15000 rpm for 60 seconds to form a semi-solid microencapsulated oil droplets (O/W emulsion) suspension. A container with 200 mL of water pre-cooled to 52° F. was placed in an ice-cooled water bath. Under stirring at 170 rpm, the O/W emulsion was transferred immediately into the container. The container was maintained in the ice-cooled water bath for 3 hours. The ice-cooled water bath was then removed, and the container was exposed to the ambient environment for another 2 hours to complete the solvent extraction step. Upon completion, the temperature of the solvent extraction was 70° F. The hardened microspheres were collected by screening through a #150 mesh sieve, washed using water through a 10 μm opening filter, and re-suspended in 20 mL of mannitol solution containing 200.0 mg mannitol for freeze-drying to obtain a powdery lyophilizate. The powdery lyophilizates were placed in a closed container at room temperature, and then stored at a −20° C. freezer. The drug loading efficiency obtained from the process is about 84.92%.

### Example 16

1561.7 mg of PLGA (75/25, 0.2 dl/g PURASORB® PDLG7502A) was dissolved in 1.3 mL of DCM with magnetic stirring to obtain a polymer solution with a concentration of 1200 mg/ml. 193.0 mg of oxybutynin was dissolved in the polymer solution, and sonication was used to dissolve oxybutynin and eliminate air from the drug-polymer solution. Then, the oxybutynin-polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 20 mL of the 1% PVA solution while homogenizing at 15000 rpm for 60 seconds to form a semi-solid microencapsulated oil droplets (O/W emulsion) suspension. Under stirring at 170 rpm, the O/W emulsion was transferred immediately into 200 mL of water for 5 hours at ambient temperature (about 70° F.) to complete the solvent extraction step. The hardened microspheres were collected by screening through a #150 mesh sieve, washed using water through a 10 μm opening filter, and re-suspended in 20 mL of mannitol solution containing 200.0 mg mannitol for freeze-drying to obtain a powdery lyophilizate. The powdery lyophilizates were placed in a closed container at room temperature, and then stored at a −20° C. freezer. The drug loading efficiency obtained from the above process is about 76.90%.

The group of Examples 15-16 of oxybutynin-loaded microspheres were prepared with the formula and procedure of Example 15 while varying the temperature of the hardening solution for the solvent extraction step. The HPLC results indicate that the drug loading efficiency was significantly influenced by the pre-cooled hardening solution applied in the solvent extraction step for preparing microspheres. The data for these examples are shown in Table 4.

TABLE 4
The drug encapsulation efficiency results from
oxybutynin microspheres prepared at the different temperatures
of the hardening solution for the extraction step.

| Examples | Example 15 | Example 16 |
|---|---|---|
| Controlled temperature of | 52-70° F. | 70° F. |
| Hardening solution (start-end) | | |
| drug loading efficiency (%) | 84.92 | 76.90 |

### Example 17

1340.5 mg of PLGA (75/25, 0.2 dl/g PURASORB® PDLG7502A) was dissolved in 4.5 mL of DCM with magnetic stirring to obtain a polymer solution with a concentration of 300 mg/ml. 370.9 mg of oxybutynin was dissolved in the polymer solution, and sonication was used to facilitate the process of dissolving oxybutynin and eliminating air from the drug-polymer solution. Then, the oxybutynin-polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 46 mL of the 1% PVA solution while homogenizing at 7000 rpm for 45 seconds to form a semi-solid microencapsulated oil droplets (O/W emulsion) suspension. Place a container with 450 mL of water pre-cooled to 45° F. into an ice-cooled water bath. Under stirring at 170 rpm, the O/W emulsion was transferred immediately into the container. The container was maintained in the ice-cooled water bath for 3 hours. The ice-cooled water bath was then removed, and the container was exposed to the ambient environment for another hour to complete the solvent extraction step. The hardened microspheres were collected by screening through a #150 mesh sieve, washed using water through a 10 μm opening filter, and re-suspended in 20 mL of mannitol solution containing 260.2 mg mannitol for freeze-drying to obtain a powdery lyophilizate. The powdery lyophilizates were placed in a closed container at room temperature, and then stored at a −20° C. freezer. The drug loading efficiency obtained from the above process is about 86.72%.

### Example 18

1341.1 mg of PLGA (75/25, 0.2 dl/g PURASORB® PDLG7502A) was dissolved in 4.5 mL of DCM with magnetic stirring to obtain a polymer solution with a concentration of 300 mg/ml; 554.7 mg of oxybutynin was dissolved in the polymer solution, and sonication was used to facilitate the process of dissolving oxybutynin and to eliminate air from the drug-polymer solution. Then, the oxybutynin-polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 46 mL of the 1% PVA solution while homogenizing at 7000 rpm for 45 seconds to form a semi-solid microencapsulated oil droplets (O/W emulsion) suspension. Place a container with 450 mL of water pre-cooled to 45° F. into an ice-cooled water bath. Under stirring at 170 rpm, the O/W emulsion was transferred immediately into the container. The container was maintained in the ice-cooled water bath for 3 hours. The ice-cooled water bath was then removed, and the container was exposed to the ambient environment for another hour to complete the solvent extraction step. The hardened microspheres were collected by screening through a #150 mesh sieve, washed using water through a 10 μm opening filter, and re-suspended in 20 mL of mannitol solution containing 260.0 mg mannitol for freeze-drying to obtain a powdery lyophilizate. The powdery lyophilizates were placed in a closed container at room temperature, and then stored at a −20° C. freezer. The drug loading efficiency obtained from the above process is about 89.50%.

Oxybutynin-loaded microspheres in Examples 13, 17, and 18 were prepared with the formula and procedure of Example 13 while varying the rate of drug (API) fed. The HPLC results are shown in Table 5.

TABLE 5
The comparison of the effect of the fed drug's rate on drug loading efficiency

| Samples | Example 13 | Example 17 | Example 18 |
|---|---|---|---|
| The rate of drug fed % | 12.5 | 21.7 | 29.3 |
| drug loading efficiency (%) | 84.42 | 86.72 | 89.5 |

Examples 19-20 below demonstrate double emulsion/solvent extraction methods.

### Example 19

1561 mg of PLGA (75/25, 0.2 dl/g PURASORB® PDLG7502A) was dissolved in 1.6 mL of DCM to obtain the polymer solution, oil phase with a concentration of 1000 mg/ml. 223.2 mg of oxybutynin HCl was dissolved in 0.25 ml of the gelatin solution (containing 28.1 mg gelatin) to obtain the drug solution, primary water phase. The oil phase was added to the primary water phase and emulsified the mixture using sonication while incubating in a water bath, and then pretreat the emulsion to 45° F. or so (being semi-solid at 45° F.) to obtain the primary emulsion, W/O emulsion. An aqueous, continuous phase that is a 1% PVA solution containing 0.01 M PBS with a pH adjusted to 8.1 was pre-cooled to 45° F. Add the primary emulsion into 25 mL of the 1% PVA solution while homogenizing at around 10000 rpm for 60 seconds to form a semi-solid microencapsulated oil droplet (W/O/W emulsion) suspension. Place a container with 300 mL of PB solution pre-cooled to 45° F. into an ice-cooled water bath. Under stirring at 170 rpm, transfer the W/O/W emulsion immediately into the container. The container was maintained in the ice-cooled water bath for 4 hours to complete the solvent extraction step, during which the temperature of the microsphere suspension rose from 50° F. to 69° F. without heating. The hardened microspheres were collected by screening through a #150 mesh sieve, washed using water through a 10 μm opening filter, and re-suspended in 15 mL of mannitol solution containing 150 mg mannitol for freeze-drying to obtain a powdery lyophilizate. The powdery lyophilizates were placed in a closed container at room temperature, and then stored at a −20° C. freezer. The drug loading efficiency obtained from the above process is about 97.71%.

### Example 20

1563.2 mg of PLGA (75/25, 0.2 dl/g PURASORB® PDLG7502A) was dissolved in 2.0 mL of DCM to obtain the polymer solution, oil phase with a concentration of 800 mg/ml. 221.6 mg of oxybutynin HCl was dissolved in 0.30 ml of the gelatin solution (containing 42.1 mg gelatin) to obtain the drug solution, primary water phase. The oil phase was added to the primary water phase, and the mixture was emulsified using sonication while incubating in a water bath. The emulsion was then pre-cooled to about 45° F. to obtain the primary emulsion (W/O emulsion). An aqueous, continuous phase that is a 1% PVA solution containing 0.01 M PBS with a pH adjusted to 8.1 was pre-cooled to 45° F. The primary emulsion was added to 30 mL of the 1% PVA solution while homogenizing at around 10000 rpm for 60 seconds to form a semi-solid microencapsulated oil droplet (W/O/W emulsion) suspension. A container with 300 mL of PBS solution pre-cooled to 45° F. was placed in an ice-cooled water bath. Under stirring at 170 rpm, the W/O/W emulsion was immediately transferred into the container. The container was maintained in the ice-cooled water bath for 4 hours to complete the solvent extraction step, during which the temperature of the microsphere suspension rose from 49° F. to 70° F. without heating. The hardened microspheres were collected by screening through a #150 mesh sieve, washed using water through a 10 μm opening filter, and re-suspended in 20 mL of mannitol solution containing 250 mg mannitol for freeze-drying to obtain a powdery lyophilizate. The powdery lyophilizates were placed in a closed container at room temperature, and then stored at a −20° C. freezer. The drug loading efficiency obtained from the above process is about 64.11%.

Oxybutynin-loaded microspheres in Examples 19 and 20 were prepared with the previously mentioned double emulsion/solvent extraction procedure while varying the concentration of the polymer solution (oil phase). The HPLC results indicate that the concentration of polymer solution (oil phase) significantly influenced the drug loading efficiency in the double emulsion technique. The results are shown in Table 6.

TABLE 6
The comparison of the effect of the concentration
of polymer solution on drug loading efficiency

| Samples | Example 19 | Example 20 |
|---|---|---|
| The concentration of oil phase (mg/ml) | 976 | 782 |
| drug loading efficiency (%) | 97.71 | 64.11 |

### Example 21

13413.9 mg of PLGA (75/25, 0.2 dl/g PURASORB® PDLG7502A) was dissolved in 19.2 mL of DCM with magnetic stirring. 1900.6 mg of oxybutynin was dissolved in the polymer solution, and sonication was used to facilitate the process of dissolving oxybutynin and eliminating air from the drug-polymer solution. Then, the oxybutynin-polymer solution was pre-cooled to 45° F. to obtain an oil phase. An aqueous, continuous phase that is a 1% PVA solution containing 0.01 M PBS with a pH adjusted to 8.1 was pre-cooled to 45° F. The oil phase was added into 200 mL of the 1% PVA solution while homogenizing at 9400 rpm for 45 seconds to form a semi-solid microencapsulated oil droplets (O/W emulsion) suspension. A container with 2000 mL of water pre-cooled to 45° F. was placed in an ice-cooled water bath. Under stirring at 250 rpm, the O/W emulsion was transferred immediately into the container. The container was maintained in the ice-cooled water bath for 3 hours. The ice-cooled water bath was then removed, and the container was exposed to the ambient environment for another hour to complete the solvent extraction step. The hardened microspheres were collected by screening through a #150 mesh sieve, washed using water through a 10 μm opening filter, and re-suspended in 80 mL of mannitol solution containing 2607.0 mg mannitol for freeze-drying to obtain a powdery lyophilizate. The powdery lyophilizates were placed in a closed container at room temperature, and then stored at a −20° C. freezer. The product yield from the above process is about 82.20%; The drug loading efficiency obtained from the above process is about 95.78%.

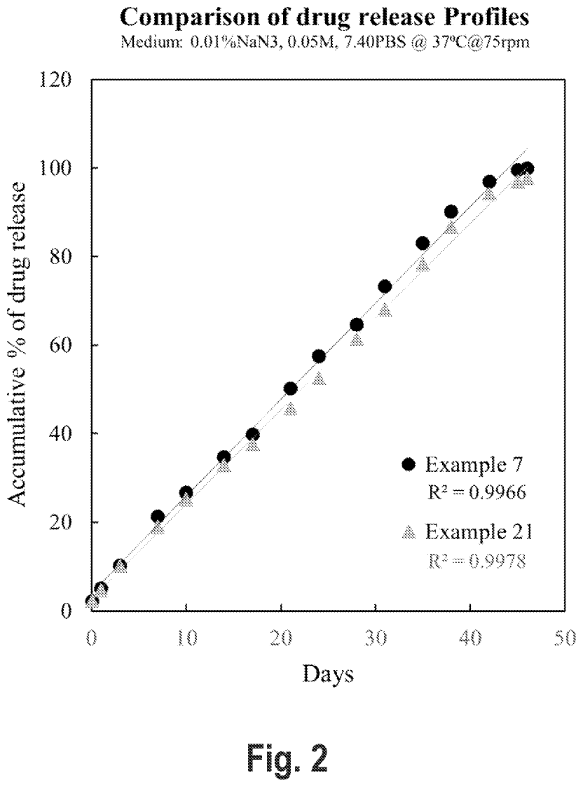

Oxybutynin-loaded microspheres in Example 21 were prepared at 10 times the scale of the currently selected formulation of Example 7. Both the product yield and the drug loading efficiency of the scaled-up batch were improved from 73.05% to 82.20% and 94.40% to 95.78%, respectively. The in vitro oxybutynin release data of both batches are shown in Table 7. The comparison of the oxybutynin-releasing characteristics of the two examples is shown in FIG. 2.

TABLE 7
A comparison of the in vitro release profiles
of the 10x scaled-up one to the original.

| Samples | Example 7 | Example 21 |
|---|---|---|
| The Product Yield (%) | 73.05 | 82.20 |
| The Drug Loading Efficiency (%) | 94.40 | 95.78 |
| Time (days) | Example 7 | Example 21 |
| 0.083 | 2 | 2 |
| 1 | 5 | 5 |
| 7 | 21 | 19 |
| 14 | 35 | 33 |
| 17 | 40 | 38 |
| 21 | 50 | 52 |
| 28 | 65 | 61 |
| 35 | 83 | 78 |
| 42 | 97 | 94 |
| 45 | 99.5 | 97 |
| 46 | 99.9 | 98 |

Examples 22-27 below, formulated for 14-18 weeks, were fabricated via a single emulsion/solvent extraction technique.

### Example 22

1342.1 mg of Poly (D.L-lactide) (0.2 dl/g PURASORB® PDL02A) was dissolved in 4.5 mL of DCM with magnetic stirring to obtain a polymer solution with a concentration of 300 mg/ml. 590.6 mg of oxybutynin was dissolved in the above polymer solution. Sonication was used to facilitate the process of dissolving Oxybutynin and eliminating air from the drug-polymer solution. Then, the Oxybutynin-polymer solution was pre-cooled to 45° F. to obtain the oil phase. The aqueous, continuous phase is a 1% PVA solution containing 0.01M PBS, pH adjusted to 8.1, and pre-cooled to 45° F. The above oil phase was added into 46 mL of the above 1% PVA while homogenizing at 4,000 RPM for 180 seconds to form the semi-solid microencapsulated oil droplets (O/W emulsion) suspension. A container with 450 mL of water precooled to 44° F. was placed into an ice-cooled water bath. While stirring at 170 rpm, the above O/W emulsion was immediately transferred into this container and maintained in the ice-cooled water bath for 3 hours, then the ice-cooled water bath was removed, and the container was maintained in the place for another hour to complete the solvent extraction step. The hardened microspheres were collected by screening through a #150 mesh sieve, washed using water through a 10 μm opening filter, and re-suspended in 20 mL of Mannitol solution containing 260.0 mg mannitol for freeze-drying to obtain a powdery lyophilizate. The powdery lyophilizates were placed in a closed container at room temperature, and then transferred into a −20° C. freezer. The above process has a product yield of 80.61%, and a drug loading efficiency of 90.92%.

### Example 23

A blend of 669.3 mg of Poly (D.L-lactide) (0.2 dl/g PURASORB® PDL 02A) and 671.9 mg of Poly (D.L-lactide-co-glycolide) (50/50, 0.2 dl/g PURASORB® PDLG 5002AY was dissolved in 4.5 mL of DCM with magnetic stirring to obtain a polymer solution with a concentration of 300 mg/ml. 561.3 mg of oxybutynin was dissolved in the above polymer solution. Sonication was used to facilitate the process of dissolving Oxybutynin and to eliminate air from the drug-polymer solution. Then, the Oxybutynin-polymer solution was pre-cooled to 45° F. to obtain the oil phase. The aqueous, continuous phase is a 1% PVA solution containing 0.01M PBS, pH adjusted to 8.1, and pre-cooled to 45° F. The above oil phase was added into 46 mL of the above 1% PVA while homogenizing at 4,000 RPM for 180 seconds to form the semi-solid microencapsulated oil droplets (O/W emulsion) suspension. A container with 450 ml of water that had been precooled to 45° F. was placed into an ice-cooled water bath. While stirring at 170 rpm, the above O/W emulsion was immediately transferred into this container. The container was maintained in the ice-cooled water bath for 3 hours, and then the ice-cooled water bath was removed. Meanwhile, the hardening solution was increased to 1,000 mL using pure water at the same temperature as the hardening solution. The container was maintained in place for another hour after the ice-cooled water bath was removed to complete the solvent extraction step. The hardened microspheres were collected by screening through a #150 mesh sieve, washed using water through a 10 μm opening filter, and re-suspended in 20 mL of Mannitol solution containing 260.0 mg mannitol for freeze-drying to obtain a powdery lyophilizate. The powdery lyophilizates were placed in a closed container at room temperature, and then transferred into a −20° C. freezer. The above process has a product yield of 87.98% and a drug loading efficiency of 92.92%.

### Example 24

A blend of 334.9 mg of Poly (D.L-lactide) (0.2 dl/g PURASORB® PDL 02A) and 1006 mg of Poly (D.L-lactide-co-glycolide) (50/50, 0.2 dl/g PURASORB® PDLG 5002AY was dissolved in 4.5 mL of DCM with magnetic stirring to obtain a polymer solution with a concentration of 300 mg/ml. 561.3 mg of oxybutynin was dissolved in the above polymer solution. Sonication was used to facilitate the process of dissolving Oxybutynin and to eliminate air from the drug-polymer solution. Then, the Oxybutynin-polymer solution was pre-cooled to 45° F. to obtain the oil phase. The aqueous, continuous phase is a 1% PVA solution containing 0.01M PBS, pH adjusted to 8.1, and pre-cooled to 45° F. The above oil phase was added into 46 mL of the above 1% PVA while homogenizing at 4,000 RPM for 180 seconds to form the semi-solid microencapsulated oil droplets (O/W emulsion) suspension. A container with 450 ml of water that has been precooled to 45° F. was placed into an ice-cooled water bath. While stirring at 170 rpm, the above O/W emulsion was immediately transferred into this container. The container was maintained in the ice-cooled water bath for 3 hours, and then the ice-cooled water bath was removed. Meanwhile, the hardening solution was increased to 1,000 mL using pure water at the same temperature as the hardening solution. The container was maintained in place for another 2 hours after the ice-cooled water bath was removed to complete the solvent extraction step. The hardened microspheres were collected by screening through a #150 mesh sieve, washed using water through a 10 μm opening filter, and re-suspended in 20 mL of Mannitol solution containing 260.0 mg mannitol for freeze-drying to obtain a powdery lyophilizate. The powdery lyophilizates were placed in a closed container at room temperature, and then transferred into a −20° C. freezer. The above process has a product yield of 83.98%, and a drug loading efficiency of 95.12%.

### Example 25

1340.0 mg of Poly (D.L-lactide-co-glycolide) (50/50, 0.2 dl/g PURASORB® PDLG 5002AY was dissolved in 4.5 mL of DCM with magnetic stirring to obtain a polymer solution with a concentration of 300 mg/ml. 561.0 mg of oxybutynin was dissolved in the above polymer solution. Sonication wasis used to facilitate the process of dissolving Oxybutynin and eliminating air from the drug-polymer solution. Then, the Oxybutynin-polymer solution was pre-cooled to 45° F. to obtain the oil phase. The aqueous, continuous phase is a 1% PVA solution containing 0.01M PBS, pH adjusted to 8.1, and pre-cooled to 45° F. The above oil phase was added into 44 mL of the above 1% PVA while homogenizing at 4,000 RPM for 180 seconds to form the semi-solid microencapsulated oil droplets (O/W emulsion) suspension. A container with 450 ml of water that has been precooled to 45° F. was placed into an ice-cooled water bath. While stirring at 170 rpm, the above O/W emulsion was immediately transferred into this container. The container was maintained in the ice-cooled water bath for 3 hours, and then the ice-cooled water bath was removed. Meanwhile, the hardening solution was increased to 1,000 mL using pure water with the same temperature as the hardening solution. The container was maintained in place for another 2 hours after the ice-cooled water bath was removed to complete the solvent extraction step. The hardened microspheres were collected by screening through a #150 mesh sieve, washed using water through a 10 μm opening filter, and re-suspended in 20 mL of Mannitol solution containing 260.0 mg mannitol for freeze-drying to obtain a powdery lyophilizate. The powdery lyophilizates were placed in a closed container at room temperature, and then transferred into a −20° C. freezer. The above process has a product yield of 89.77% and a drug loading efficiency of 95.20%.

### Example 26

A blend of 669.5 mg of Poly (D.L-lactide) (0.2 dl/g PURASORB® PDL 02A) and 673.2 mg of Poly (D.L-lactide-co-glycolide) (75/25, 0.2 dl/g PURASORB® PDLG 7502A was dissolved in 4.5 mL of DCM with magnetic stirring to obtain a polymer solution with a concentration of 300 mg/ml. 562.3 mg of oxybutynin was dissolved in the above polymer solution. Sonication was used to facilitate the process of dissolving Oxybutynin and to eliminate air from the drug-polymer solution. Then, the Oxybutynin-polymer solution was pre-cooled to 45° F. to obtain the oil phase. The aqueous, continuous phase is a 1% PVA solution containing 0.01M PBS, pH adjusted to 8.1, and pre-cooled to 45° F. The above oil phase was added into 46 mL of the above 1% PVA while homogenizing at 4,000 RPM for 180 seconds to form the semi-solid microencapsulated oil droplets (O/W emulsion) suspension. A container with 450 ml of water precooled to 45° F. was placed into an ice-cooled water bath. While stirring at 170 rpm, the above O/W emulsion was immediately transferred into this container. The container was maintained in the ice-cooled water bath for 3 hours, and then the ice-cooled water bath was removed. Meanwhile, the hardening solution was increased to 1,000 mL using pure water at the same temperature as the hardening solution. The container remained in place for another hour after the ice-cooled water bath was removed to complete the solvent extraction step. The hardened microspheres were collected by screening through a #150 mesh sieve, washed using water through a 10 μm opening filter, and re-suspended in 20 mL of Mannitol solution containing 260.0 mg mannitol for freeze-drying to obtain a powdery lyophilizate. The powdery lyophilizates were placed in a closed container at room temperature, and then transferred into a −20° C. freezer. The above process has a product yield of 86.46% and a drug loading efficiency of 90.12%.

### Example 27

A blend of 335.9 mg of Poly (D.L-lactide) (0.2 dl/g PURASORB® PDL 02A) and 1005.6 mg of Poly (D.L-lactide-co-glycolide) (75/25, 0.2 dl/g PURASORB® PDLG 7502A was dissolved in 4.5 mL of DCM with magnetic stirring to obtain a polymer solution with a concentration of 300 mg/ml. 564.9 mg of oxybutynin was dissolved in the above polymer solution. Sonication was used to facilitate the process of dissolving Oxybutynin and eliminating air from the drug-polymer solution. Then, the Oxybutynin-polymer solution was pre-cooled to 45° F. to obtain the oil phase. The aqueous, continuous phase is a 1% PVA solution containing 0.01M PBS, pH adjusted to 8.1, and pre-cooled to 45° F. The above oil phase was added into 46 mL of the above 1% PVA while homogenizing at 4,000 RPM for 180 seconds to form the semi-solid microencapsulated oil droplets (O/W emulsion) suspension. A container with 450 mL of water precooled to 45° F. was placed into an ice-cooled water bath. While stirring at 170 rpm, the above O/W emulsion was immediately transferred into this container. The container was maintained in the ice-cooled water bath for 3 hours, and the ice-cooled water bath was then removed. Meanwhile, the hardening solution was increased to 1,000 mL using pure water at the same temperature as the hardening solution. The container remained in place for another 2 hours after the ice-cooled water bath was removed to complete the solvent extraction step. The hardened microspheres were collected by screening through a #150 mesh sieve, washed using water through a 10 μm opening filter, and re-suspended in 20 mL of Mannitol solution containing 260.0 mg mannitol for freeze-drying to obtain a powdery lyophilizate. The powdery lyophilizates were placed in a closed container at room temperature, and then transferred into a −20° C. freezer. The above process has a product yield of 80.50% and a drug loading efficiency of 89.30%.

All samples of Oxybutynin loaded microspheres, as described in Examples 22-27, were prepared using the formula and procedure described in Example 22, while varying the ratios of PLA to PLGA. The size of the microspheres in all batches ranges from 10 to 75 μm. For evaluating the effects of the frequency and/or the length of product samples “quenched” after freeze-drying, in vitro drug release was conducted for three sets of samples prepared according to Examples 23 and 25 and two sets of samples prepared according to Examples 26 and 27. The in vitro release data of the samples of Examples 23 and 25 are shown in Table 8, and those of the samples of Examples 26 and 27 are shown in Table 9.

The results indicate that the frequency and/or the length of “quenching” the powdery lyophilizates significantly lowered the burst on the first day and influenced the entire profile (e.g., the releasing period). Further, the results show that the frequency and/or duration of “quenching” the powdery lyophilizates required to acquire the desired release characteristics of microsphere products depend on the ratio of PLGA to PLA.

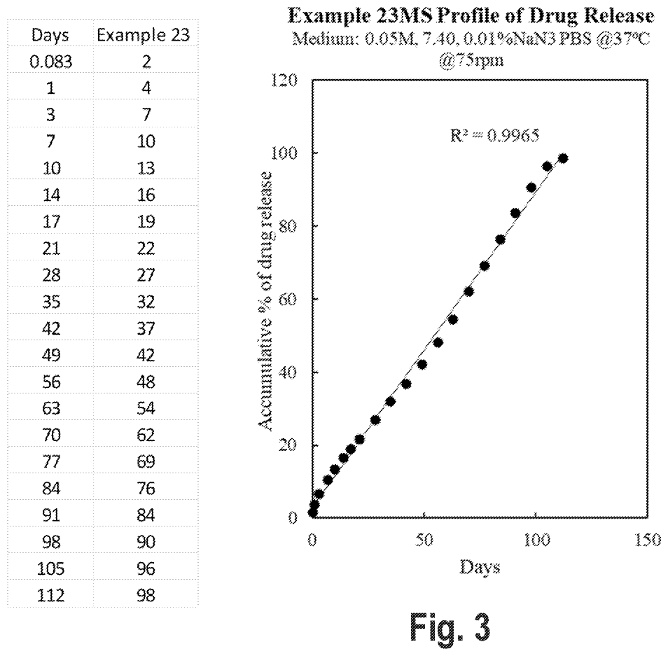

In this group of examples, the desired in vitro drug release data of the samples of Examples 22-27 are shown in Table 10. The formulation of Example 23 (quenched once in a 3-week period) was selected as an optimal formulation due to the product yield, percent of encapsulation efficiency, the burst on the first day of drug release from microspheres, and the overall release characteristics of the formulation. The Oxybutynin release characteristics of the samples of Example 23 in the in vitro study are shown in FIG. 3

TABLE 8
Comparison of the effects of quenching microsphere products
on the drug release profiles in the in vitro study.

| | Example | Example | Example | Example | Example | Example |
|---|---|---|---|---|---|---|
| | 23 (%) | 23 (%) | 23 (%) | 25 (%) | 25 (%) | 25 (%) |
| Time | No | Quenched | Quenched | No | Quenched | Quenched |
| (days) | Quenching | Once | Twice | Quenching | Once | Twice |
| 0.083 | 2.0 | 1.6 | 1.9 | 1.4 | 1.4 | 1.4 |
| 1 | 6.4 | 3.7 | 3.9 | 7.9 | 5.2 | 4.2 |
| 3 | 11.1 | 6.6 | 6.8 | 15.6 | 11.8 | 9.7 |
| | | | | (d5) | | |
| 7 | 16.1 | 10.5 | 11.5 | 19.4 | 16.2 | 14.4 |
| | (d6) | | | | | |
| 10 | 19.6 | 13.4 | 14.1 | 22.2 | 19.3 | 17.2 |
| | (d9) | | | | | |
| 14 | | 16.4 | 16.9 | | 22.0 | 19.9 |
| 17 | | 18.9 | 19.2 | | 25.1 | 21.8 |
| 21 | | 21.6 | 21.8 | | 28.3 | 24.8 |
| 28 | | 26.9 | 26.1 | | 35.7 | 30.7 |
| 35 | | 31.8 | 30.0 | | 42.4 | 37.6 |
| 42 | | 36.9 | 35.8 | | 49.8 | 44.2 |
| 49 | | 42.0 | 42.3 | | 57.2 | 50.0 |
| 56 | | 48.1 | 49.3 | | 64.2 | 56.3 |
| 63 | | 54.3 | 56.3 | | 70.8 | 62.3 |
| 70 | | 62.0 | 63.5 | | 76.6 | 67.6 |
| 77 | | 69.1 | 69.7 | | 82.2 | 72.8 |
| 84 | | 76.3 | 75.0 | | 87.2 | 77.9 |
| 91 | | 83.6 | 80.8 | | 91.8 | 82.2 |
| 98 | | 90.4 | 86.6 | | 96.0 | 86.8 |
| 105 | | 96.3 | 90.2 | | 97.0 | 90.1 |
| | | | | | (d101) | |
| 112 | | 98.4 | 93.7 | | | 93.8 |
| 119 | | | 96.4 | | | 97.0 |
| 122 | | | 97.4 | | | 97.9 |
| Forced | | 100.0 | 100.0 | | 100.0 | 100.0 |
| re1ease) | | (d112) | (d122) | | (d101F) | (d122F) |
| recovered | | | | | | |

As shown in Table 8, the results indicate that the frequency and duration of quenching microsphere products significantly influenced the initial burst on the first day and the entire drug release characteristics of PLGA/PLA-based microsphere products.

TABLE 9
A comparison of the effects of quenching microsphere products
on the drug release profiles in the in vitro study.

| | Example 26 | Example 26 | Example 27 | Example 27 |
|---|---|---|---|---|
| | (%) | (%) | (%) | (%) |
| | Quenched | Quenched | Quenched | Quenched |
| Time (days) | Once | Twice | Once | Twice |
| 0.083 | 3.9 | 3.1 | 2.7 | 2.5 |
| 1 | 8.7 | 5.2 | 7.2 | 5.8 |
| 7 | 18.2 | 11.5 | 17.0 | 13.5 |
| 14 | 27.2 | 16.9 | 23.5 | 18.8 |
| 17 | 31.5 | 19.8 | 25.9 | 21.7 |
| | (d18) | | | (d18) |
| 21 | 35.2 | 22.1 | 28.8 | 23.9 |
| 28 | 42.5 | 27.1 | 34.0 | 28.9 |
| 35 | 48.6 | 31.6 | 39.4 | 32.4 |
| 42 | | 373 | 45.0 | 36.5 |
| 49 | | 42.5 | 51.4 | 41.2 |
| 56 | | 49.6 | 57.9 | 47.6 |
| 63 | | 57.1 | 64.4 | 54.5 |
| 70 | | 64.1 | 70.3 | 60.6 |
| 77 | | 71.0 | 76.8 | 65.5 |
| 84 | | 78 | 82.6 | 70.7 |
| 91 | | 84.7 | 88.9 | 75.7 |
| 98 | | 91.6 | 93.9 | 82.4 |
| 105 | | 96.0 | 96.6 | 88.0 |
| | | | | (d104) |
| 108 | | 98.0 | 98.2 | 91.9 |
| | | | | (d109) |
| 119 | | 99.0 | | 96.8 |
| | | (d112) | | |
| 125 | | | | 98.7 |
| (Forced release) | | 100.9 | 98.2 | 99.9 |
| recovered | | (d112F) | (d108F) | (d125F) |

The results show that the frequency and duration of quenching microsphere products significantly influenced the initial burst on the first day and the entire drug release characteristics of PLGA/PLA-based microsphere products.

TABLE 10
The desired in vitro drug release results of the Oxy. Microspheres prepared at the different rates of PLGA/PLA.

| | Example | Example | Example | Example | Example | Example |
|---|---|---|---|---|---|---|
| | 22 (%) | 23 (%) | 24 (%) | 25 (%) | 26 (%) | 27 (%) |
| Time | No | Quenched | Quenched | Quenched | Quenched | Quenched |
| (days) | Quenching | Once | Once | Once | Twice | Twice |
| 1 | 5.2 | 3.7 | 3.1 | 5.2 | 5.2 | 5.8 |
| 3 | 8.3 | 6.6 | 7.1 | 11.8 | 8.1 | 9.6(d4) |
| 7 | 11.5 | 10.5 | 10.9 | 16.2 | 11.5 | 13.5 |
| 10 | 13.1 | 13.4 | 13.3 | 19.3 | 14.2 | 16.3(d11) |
| 14 | 15.9 | 16.4 | 16.2 | 22.0 | 16.9 | 18.8 |
| 17 | 18.3 | 18.9 | 18.8 | 25.1 | 19.8 | 21.7(d18) |
| 21 | 20.8 | 21.6 | 21.3 | 28.3 | 22.1 | 23.9 |
| 28 | 25.1 | 26.9 | 25.9 | 35.7 | 27.1 | 28.9 |
| 35 | 29.7 | 31.8 | 31.0 | 42.4 | 31.6 | 32.4 |
| 42 | 37.3 | 36.9 | 37.5 | 49.8 | 37.3 | 36.5 |
| 49 | 44.7 | 42.0 | 44.0 | 57.2 | 42.5 | 41.2 |
| 56 | 51.9 | 48.1 | 51.2 | 64.2 | 49.6 | 47.6 |
| 63 | 60.0 | 54.3 | 58.3 | 70.8 | 57.1 | 54.5 |
| 70 | 67.6 | 62.0 | 64.9 | 76.6 | 64.1 | 60.6 |
| 77 | 74.7 | 69.1 | 71.3 | 82.2 | 71.0 | 65.5 |
| 84 | 81.9 | 76.3 | 77.3 | 87.2 | 78.0 | 70.7 |
| 91 | 88.6 | 83.6 | 81.7 | 91.8 | 84.7 | 75.7 |
| 98 | 93.8 | 90.4 | 86.1 | 96.0 | 91.6 | 82.4 |
| 105 | 95.0(d100) | 96.3 | 90.6 | 97.0(d101) | 96.0 | 88.0(d104) |
| 112 | 95.7(d101) | 98.4 | 93.1 | | 99.0 | 93.7 |
| 115 | | | 94.7 | | | 95.4(d116) |
| 119 | | | | | | 96.8 |
| 125 | | | | | | 98.7 |
| (Forced re1ease) | 100.0(d101F) | 100.0(d112F) | 100.0(d115F) | 100.0(d101F) | 100.9(d112F) | 99.9(d125F) |
| recovered | | | | | | |

The results indicate that the frequency and/or duration of “quenching” the powdery lyophilizates required to acquire the desired release characteristics of microsphere products depend on the ratio of PLGA to PLA.

### Microsphere Product's Stability

To evaluate the stability of microsphere products, we measured the API amount in a few products at different time points using the assay method. All the samples were stored in a −20° C. freezer. The assay results indicate that the microsphere product is stable in a 30-month period under the current conditions. The assay results of Example 7 at 4 months, 8 months, and 30 months are shown on table 11; that of example 22-25 at day 0 and 6 months on table 12.

TABLE 11
The stability study assay results of Example 7 at 4, 8, and 30 months.

| The Product Yield (%) | 73.05 |
|---|---|
| Drug Loading Efficiency (%) | 94.4 |
| Time (months) | Assay % |
| 4 | 9.39 |
| 8 | 9.45 |
| 30 | 9.39 |

The assay results show that the microsphere product prepared according to Example 7 is stable for at least 30 months.

TABLE 12
The stability study assay results of Examples 22-25 at 0 and 6 months.

| | Example 22 | Example 23 | Example 24 | Example 25 |
|---|---|---|---|---|
| The Product Yield (%) | 80.61 | 87.98 | 83.98 | 89.77 |
| The Drug Loading Efficiency (%) | 90.92 | 92.92 | 95.12 | 95.20 |
| Time (months) | Assay % | Assay % | Assay % | Assay % |
| 0 | 22.88 | 22.23 | 22.05 | 22.45 |
| 6 | 22.54 | 21.83 | 22.77 | 22.61 |

### Example 28

524.2 mg of Poly (D.L-lactide-co-glycolide) (75/25, 0.2 dl/g PURASORB® PDLG75016A) and 1569.3 mg Poly (D.L-lactide-co-glycolide) (50/50, 0.2 dl/g PURASORB® PDLG5002AY) was dissolved in 2.4 mL of DCM to obtain the polymer solution, oil phase with a concentration of 900 mg/ml. 230.5 mg of Pramipexole 2HCl H₂O was dissolved in 0.30 ml of the gelatin solution (containing 138 mg gelatin) to obtain the drug solution, primary water phase. The above oil phase was added into the primary water phase, and the mixture was emulsified using sonication while incubating in a water bath, and then the emulsion was pretreated to about 45° F. to obtain the primary emulsion, W/O emulsion. The aqueous, continuous phase is a 1% PVA solution containing 0.01M PBS, pH adjusted to 8.0, and pre-cooled to 45° F. Add the above primary emulsion into 30 mL of the above 1% PVA while homogenizing at around 12000 RPM for 80 seconds to form the semi-solid microencapsulated oil droplets (W/O/W emulsion) suspension. A container with 300 mL of PB (0.2M, pH 3.0) solution and precooled to 45° F. was provided. Under stirring at 300 rpm, the above W/O/W emulsion was transferred immediately into this container at ambient environment for 3 hours to complete the solvent extraction step, during which the temperature of the microsphere suspension rose from 50° F. to 70° F. without heating. A portion of the hardened microspheres was collected by screening through a #150 mesh sieve and centrifuged at 4000 g for 3 minutes, washed using 200 ml 0.2M, pH 3.0 PB solution, and then re-suspended in 10 mL of Mannitol solution containing 100 mg mannitol for freeze-drying to obtain a powdery lyophilizate. The powdery lyophilizates were placed in a closed container at room temperature, and then stored at a −20° C. freezer. The process above resulted in a drug loading efficiency of 83.73%.

### Example 29

1039.6 mg of Poly (D.L-lactide-co-glycolide) (75/25, 0.2 dl/g PURASORB® PDLG75016A) and 1066.5 mg Poly (D.L-lactide-co-glycolide) (50/50, 0.2 dl/g PURASORB® PDLG5002AY) was dissolved in 2.4 mL of DCM to obtain the polymer solution, oil phase with a concentration of 900 mg/ml. 232.2 mg of Pramipexole 2HCl H₂O was dissolved in 0.30 ml of the gelatin solution (containing 28.2 mg gelatin) to obtain the drug solution, primary water phase. The above oil phase was added into the primary water phase, and the mixture was emulsified using sonication while incubating in a water bath, and then the emulsion was pretreated to about 45° F. to obtain the primary emulsion, W/O emulsion. The aqueous, continuous phase is a 1% PVA solution containing 0.01M PBS, pH adjusted to 8.0, and pre-cooled to 45° F. The above primary emulsion was added into 30 mL of the above 1% PVA while homogenizing at around 12000 RPM for 80 seconds to form the semi-solid microencapsulated oil droplets (W/O/W emulsion) suspension. A container with 300 mL of PB (0.2 M, pH 3.0) solution that had been precooled to 45° F. was provided. Under stirring at 300 rpm, the above W/O/W emulsion was transferred immediately into this container at ambient environment for 3 hours to complete the solvent extraction step, during which the temperature of the microsphere suspension rose from 50° F. to 70° F. without heating. A portion of the hardened microspheres was collected by screening through a #150 mesh sieve and centrifuge at 4000 g for 3 minutes, washed using 100 ml pure water, and then re-suspended in 10 mL of Mannitol solution containing 100 mg mannitol for freeze-drying to obtain a powdery lyophilizate. The powdery lyophilizates were placed in a closed container at room temperature, and then stored at a −20° C. freezer. The above process resulted in a drug loading efficiency of 72.7%.

### Example 30

2508.1 mg of Poly (D.L-lactide-co-glycolide) (75/25, 0.2 dl/g PURASORB® PDLG75016A) was dissolved in 2.3 mL of DCM to obtain the polymer solution, oil phase with a concentration of 1100 mg/ml. 273 mg of Pramipexole 2HCl H2O was dissolved in 0.22 ml of the gelatin solution (containing 31 mg gelatin) to obtain the drug solution, primary water phase. the above oil phase was added into the primary water phase, and the mixture was emulsified using sonication while incubating in a water bath, and then the emulsion was pretreated to about 45° F. obtain the primary emulsion, W/O emulsion. The aqueous, continuous phase is a 1% PVA solution containing 0.01M PBS, pH adjusted to 8.0, and pre-cooled to 45° F. The above primary emulsion was added into 30 mL of the above 1% PVA while homogenizing at around 12000 RPM for 70 seconds to form the semi-solid microencapsulated oil droplets (W/O/W emulsion) suspension. A container with 300 mL of PB (0.2M, pH 3.0) solution that had been precooled to 40° F. was provided. Under stirring at 300 rpm, the above W/O/W emulsion was transferred immediately into this container at ambient environment for 3 hours to complete the solvent extraction step, during which the temperature of the microsphere suspension rose from 50° F. to 70° F. without heating. The hardened microspheres were collected by screening through a #150 mesh sieve and centrifuge at 4000 g for 3 minutes, washed using 100 ml pure water, and then re-suspended in 10 mL of Mannitol solution containing 100 mg mannitol for freeze-drying to obtain a powdery lyophilizate. The powdery lyophilizates were placed in a closed container at room temperature, and then stored it at a −20° C. freezer. The above process resulted in a drug loading efficiency of 68.86%.

All samples of Examples 28-30 of Pramipexole Dihydrochloride loaded microspheres were prepared with the previously mentioned formula and procedure except the ratio of LA to GA, or I.V of the PLGA and concentration of the polymer solution. The size of the microspheres in all batches range from 5 μm to 75 μm.

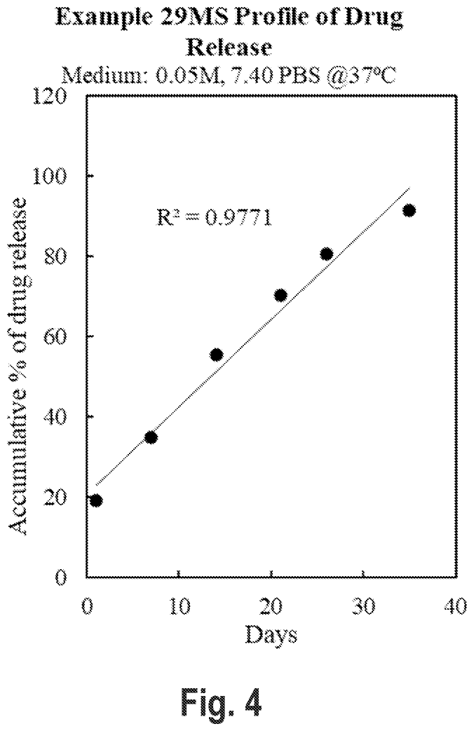

In this group of examples, the formulation of Example 29 was selected as the currently optimal formulation due to size, size distribution and shape of microspheres, % of encapsulation efficiency, % of drug release from microspheres, and the overall release characteristics of the formulation. The in vitro release data for these samples are shown in Table 13. The Pramipexole release characteristics of the sample of Example 29 in the in vitro study is shown in FIG. 4

TABLE 13
The drug release results of the in vitro of Pramipexole
Microspheres prepared@ the Diff. contents of PLGA via
the double emulsion/solvent extraction technique.

| Time (days) | Example 28 | Example 29 | Example 30 |
|---|---|---|---|
| 1 | 15.2 | 19.3 | 42.2 |
| 7 | 25.1 | 34.9 | 43.8 |
| 14 | 32.6 | 55.4 | 56 |
| | (d 16) | | |
| 21 | 30.9 | 70.2 | 64.8 |
| 26 | | 80.7 | 68.6 |
| | | | (d28) |
| 35 | | 91.4 | 75.7 |
| 42 | | 85.3 | 90.1 |
| 49 | | 83.7 | |

### Determination of Drug Loading Efficiency for Pramipexole Microspheres

About 20 mg of microspheres were added to 3 mL DCM using the vortex for 20 seconds before and after a 20-minute interval. 2 ml water was added into the suspension to extract Pramipexole and then vortexed until the mixture became clear; 1 ml supernatant was added to 1 ml pure water and mixed well. The samples were filtered through a 0.45 μm PTFE syringe filter and then analyzed by HPLC to measure the amount of Pramipexole.

The percentage of loading efficiency was calculated by the tested drug loading rate to the theoretical rate of the drug entrapment.

Alternatively, it can be calculated by [the amount of feeding API-the amount of API (lost in hardening Sol.)]/the amount of feeding API *100%.

The Agilent HPLC conditions are as follows:
Column: Zorbax Eclipse XDB-C18 4.6×150 mm (5 μm)
Mobile Phase consists of Phosphate Buffer/Sodium I-Octane and ACN=50:50.
Running time: 10 minutes
The flow rate: 1 mL/min
Injection vol. 20 μL and the detection wavelength: 262 nm.

### The In Vitro Release Method for Pramipexole Microspheres

About 20 mg of the formulated Microspheres were weighed into a 7 ml glass vial with screw cap filled with 5 mL PBS (0.05M, pH 7.4) and immersed into a water bath at 37° C. The release medium was replaced at the following time points: 2 hours, days 1, 7, 14, 21, 26, 35, until release is completed and then filtered through a 0.45 μm opening PES syringe filter for HPLC analysis.

The Agilent HPLC conditions are as follows:
Column: Zorbax Eclipse XDB-C18 4.6×150 mm (5 μm)
Mobile Phase consists of Phosphate Buffer/Sodium I-Octane and ACN=50:50.
Running time: 10 minutes
The flow rate: 1 mL/min
Injection vol. 20 μL and the detection wavelength: 262 nm.

The present disclosure is not to be limited in scope by the specific embodiments described herein. Indeed, various modifications of the invention, in addition to those described herein, will become apparent to those skilled in the art from the foregoing description and the accompanying figures. Such modifications are intended to fall within the scope of the appended claims.

What is claimed is:

1. A microsphere for controlled long-term sustained delivery of an active agent, comprising the active agent and a polymer carrier that encapsulates the active agent, wherein the polymer carrier comprises polylactide (PLA), polyglycolic acid (PLG), poly(lactide-co-glycolide) (PLGA), polyethylene glycol-PLA, PLA-polycaprolactone (PCL), a polyorthoester, a polyphosphazene, a polyphosphoester, or a combination thereof.

2. The microsphere of claim 1, wherein the polymer carrier comprises PLGA, PLA, or a combination thereof.

3. The microsphere of claim 1, wherein the microsphere has a diameter of from about 8 μm to about 75 μm.

4. The microsphere of claim 1, wherein the microsphere comprises from about 4% (w/w) to about 50% (w/w) of the active agent by weight of the microsphere.

5. The microsphere of claim 1, wherein the polymer carrier is biodegradable and biocompatible.

6. The microsphere of claim 1, wherein the polymer carrier has a degradation half-life of at least 2 months under physiological conditions.

7. The microsphere of claim 1, wherein the microsphere has a zero-order release when contacting with an aqueous phase.

8. The microsphere of claim 1, wherein the microsphere maintains a controlled and sustained release for a period from 1 week to 6 months.

9. The microsphere of claim 1, wherein the active agent comprises xybutynin, tolterodine, solifenacin, darifenacin, pramipexole, or tamsulosin.

10. The microsphere of claim 9, wherein the active agent comprises a free base, a stereoisomer, a derivative, an analog, a prodrug, or a pharmaceutically acceptable salt of oxybutynin, tolterodine, solifenacin, darifenacin, pramipexole, or tamsulosin.

11. A composition, comprising the microsphere of claim 1.

12. The composition of claim 11, further comprising at least one anti-cryogenic agent.

13. The composition of claim 12, further comprising an excipient.

14. The composition of claim 13, further comprising an additional therapeutic agent.

15. The composition of claim 14, wherein the additional therapeutic agent comprises an α2δ subunit calcium channel modulator, a β3 adrenergic agonist, a spasmolytic, a neurokinin receptor antagonist, a bradykinin receptor antagonist, a nitric oxide donor, or a combination thereof.

16. A kit or an implant, comprising the microsphere of claim 1.

17. A method of preparing a microsphere according to claim 1, comprising:
(a) dissolving PLGA in at least one organic solvent that is substantially non-miscible with water to form a polymer solution;
(b) dissolving an active agent in the polymer solution by single emulsion/solvent extraction to obtain an oil phase drug-polymer solution, or by double emulsion to obtain a water-in-oil (W/O) primary emulsion, and cooling the oil phase drug-polymer solution or the water-in-oil primary emulsion to about 45° F.;
(c) adding the oil phase drug-polymer solution or the water-in-oil primary emulsion to a PVA solution, wherein the PVA solution is in the form of an aqueous, continuous phase;
(d) homogenizing the oil phase drug-polymer solution or the water-in-oil primary emulsion with the 1% PVA solution at a predetermined mixing rate to obtain a microsphere suspension having semi-solid microencapsulated droplets, wherein the microsphere suspension is in the form of oil-in-water or water-in-oil-in-water (W/O/W) emulsion;
(e) extracting the organic solvent by:
placing a container containing water precooled to about 45° F. in an ice-cooled water bath,
transferring the microsphere suspension into the container while stirring the microsphere suspension with a mixer,
maintaining the container in the ice-cooled water bath for about 3 hours to allow the microsphere suspension to harden and the temperature thereof to rise from about 45° F. to about 55° F., and
removing the ice-cooled water bath and exposing the microsphere suspension to an ambient temperature without heating equipment for about 1 hour, such that the temperature of the hardened microsphere suspension reaches about 66° F.; and
(f) collecting microspheres by screening through a mesh sieve, washing with water through a filter, and re-suspending in a D-mannitol solution for freeze-drying to obtain powdery lyophilized microspheres.

18. The method of claim 17, further comprising: after step (f), quenching the powdery lyophilized microspheres one or more times at a temperature below 25 degrees Celsius.

19. The method of claim 18, wherein the step of quenching is performed by placing the powdery lyophilized microspheres at −20 degrees Celsius.

20. The method of claim 17, wherein the organic solvent comprises dichloromethane.

21. The method of claim 17, wherein the polymer solution contains from about 50 mg/mL to about 1200 mg/mL of PLGA.

22. The method of claim 17, wherein the PVA solution contains from about 0.1% to about 5% (w/v) of PVA.

23. The method of claim 22, wherein the PVA solution contains about 1% (w/v) of PVA.

24. The method of claim 17, wherein the PVA solution contains a buffer having a concentration of from about 0.0001 M to about 1 M.

25. The method of claim 24, wherein the buffer has a concentration of about 0.01 M.

26. The method of claim 17, wherein the buffer has a pH of about 1 to about 14.

27. The method of claim 26, wherein the buffer has a pH of about 8.1.

28. The method of claim 17, wherein the buffer is a phosphate-buffered saline (PBS) buffer.

29. The method of claim 17, wherein the PVA solution at step (c) has a temperature of from about 35° F. to 73° F.

30. The method of claim 29, wherein the PVA solution at step (c) has a temperature of about 45° F.

31. The method of claim 17, wherein the predetermined mixing rate is from about 2000 rpm to about 20000 rpm.

32. The method of claim 17, wherein an aqueous phase in step (e) has a volume at least 2 times greater than that of an oil/water phase.

33. The method of claim 32, wherein the aqueous phase in step (e) comprises a buffer having a concentration of from about 0.001 M to about 2 M.

34. The method of claim 33, wherein the buffer has a pH of from about 1 to about 14.

35. The method of claim 17, wherein during step (e), the temperature of the microsphere suspension rises from about 35° F. to the ambient temperature.

36. The method of claim 17, wherein the ambient temperature is about 73° F.

37. The method of claim 17, wherein: (i) when the container is in the ice-cooled water bath, the temperature of the microsphere suspension gradually increases as that of the ice-cooled water bath does, and (ii) when the iced-cooled water bath is removed, the temperature of the microsphere suspension continues to gradually increase towards the ambient temperature.

38. The method of claim 17, wherein the microspheres have an average particle size of from about 1 μm to about 300 μm.

39. The method of claim 17, wherein the active agent comprises oxybutynin, tolterodine, solifenacin, darifenacin, pramipexole, or tamsulosin.

40. The method of claim 39, wherein the active agent comprises a free base, a stereoisomer, a derivative, an analog, a prodrug, or a pharmaceutically acceptable salt of oxybutynin, tolterodine, solifenacin, darifenacin, pramipexole, or tamsulosin.
